(19) United States
(12) Patent Application Publication
(10) Pub. No.: US 2019/0388364 A1
(43) Pub. Date: Dec. 26, 2019

(54) TAPENTADOL AS A LOCAL ANESTHETIC

(71) Applicant: GRÜNENTHAL GMBH, Aachen (DE)

(72) Inventors: Johannes SCHNEIDER, Stolberg (DE); Thomas CHRISTOPH, Aachen (DE)

(73) Assignee: GRÜNENTHAL GMBH, Aachen (DE)

(21) Appl. No.: 16/545,567

(22) Filed: Aug. 20, 2019

Related U.S. Application Data
(63) Continuation of application No. PCT/EP2018/054327, filed on Feb. 22, 2018.

(30) Foreign Application Priority Data
Feb. 23, 2017 (EP) 17157584.8

(51) Int. Cl. A61K 31/137; A61K 9/00; A61K 47/12

(52) U.S. Cl. CPC A61K 31/137; A61K 47/12; A61K 9/0019; A61K 9/0014

(57) ABSTRACT

The invention relates to a pharmaceutical composition comprising Tapentadol or a physiologically acceptable salt thereof for use in local anesthesia.

This application is a continuation of PCT International Application No. PCT/EP2018/054327, filed Feb. 22, 2018, which claims foreign priority benefit under 35 U.S.C. § 119 of European Patent Application No. 17157584.8, filed Feb. 23, 2017, the disclosures of each of which are incorporated herein by reference.

The invention relates to a pharmaceutical composition comprising Tapentadol or a physiologically acceptable salt thereof for use in local anesthesia.

Tapentadol is a centrally acting opioid analgesic of the benzenoid class with a dual mode of action as an agonist of the μ-opioid receptor and as a norepinephrine reuptake inhibitor (NRI).

Pharmaceutical dosage forms of Tapentadol are known from the prior art, e.g., WO 02/67651, WO 03/035053, WO 2006/002886, WO 2007/128412, WO 2007/128413, WO 2008/110323, WO 2009/067703, WO 2009/092601, US 2010/272815, and T. M. Tzschentke et al., Drugs of the future, 31(12), 2006, 1053-1061.

Anesthesia differs from analgesia. While analgesia aims at abolishing pain sensation, anesthesia may be regarded as a state of local or general insensitiveness for the purpose of surgery or a diagnostic measure. Insensitiveness in anesthesia is typically not only to pain, but to various stimuli including pain stimuli, temperature stimuli, contact stimuli, and the like. In analgesia, such general insensitiveness to various stimuli is not desirable because of the risk that the subject unintentionally hurts itself. For example, after a medical intervention in the mouth requiring local anesthesia, it usually takes some time until local anesthesia has declined. During this period, the subject is a risk to hurt itself unintentionally, e.g. by biting into the lips or the tongue.

The prefix “an-” of the word “analgesia” means “without”, whereas the suffix “-algesia” means “pain, sensitivity”. Analgesia thus means a lack of sensations, a deadening or absence of the sense of pain without the loss of consciousness. A main pharmacological action of an analgesic (painkiller, pain reliefer) can be on the cerebrum and medulla of the central nervous system.

The prefix “an-” of the word “anesthesia” means “without”, whereas the suffix “-esthesia” means “feeling, sensation”. Anesthesia thus means local or general insensibility to pain with or without the loss of consciousness, induced by an anesthetic. There are four features of anesthesia (sedation): (i) lack of motor response to instructions; (ii) suppression of autonomic and skeletal response to intraoperative stimuli such as incisions; (iii) absence of retrospective awareness of pain; and (iv) postoperative amnesia for surgical events such as conversations among medical team.

Major differences between analgesia and anesthesia can include: (i) analgesia is a lack of pain, whereas anesthesia refers to a lack of sensation; (ii) anesthesia can be accompanied by analgesia; (iii) patients are fully aware and awake while using analgesia, whereas with anesthesia patients can be either unconscious and asleep, or awake and fully comprehensible; and (iv) analgesia is patient controlled, whereas anesthesia is controlled by a person specializing in administration of anesthesia.

Thus, the clinical situation of using Tapentadol in anesthesia, especially in local anesthesia, significantly differs from the clinical situation of using Tapentadol in analgesia such as local analgesia.

An anesthetic is a pharmacologically active compound to prevent pain during surgery or a diagnostic measure. A wide variety of pharmacologically active compounds are used in modern anesthetic practice. Anesthetics are categorized into two classes: general anesthetics, which cause a reversible loss of consciousness, and local anesthetics, which cause a reversible loss of sensation for a limited region of the body while maintaining consciousness. Each of the local anesthetics have the suffix “-caine” in their names, e.g. amethocaine, bupivacaine, dibucaine, levobupivacaine, lidocaine, mepivacaine, prilocaine, procaine, and ropivacaine.

B Ramanath Royal, Int J Pharm Bioscience, 3(1), 2012, 479-484 discloses Tapentadol as μ-opioid agonist useful in the treatment of pain. The local administration of Tapentadol is not disclosed.

CN 103 735 500 discloses aqueous compositions comprising Tapentadol useful for treating pain. The local administration of Tapentadol is not disclosed.

WO 2012/119727 relates to an aqueous pharmaceutical composition containing Tapentadol or a physiologically acceptable salt thereof and being adapted for oral administration. The composition has excellent storage stability without relying on the presence of high amounts of preservatives.

WO 2012/119728 discloses parenteral formulations for the administration of Tapentadol. The concentration of Tapentadol in these formulations is preferably below 100 mg/mL. The concentration of Tapentadol in the exemplified formulations according to WO 2012/119728 is 15 mg/mL and 20 mg/mL, respectively. According to WO 2012/119728 Tapentadol exhibits antimicrobial properties. These antimicrobial properties are more pronounced at higher pH values. In consequence, preservatives may be omitted or their content in the formulations may at least be decreased. The complete absence of preservatives is preferred when the content of Tapentadol is sufficiently high so that due to its preserving property the desired shelf life or in use stability can be achieved by the presence of Tapentadol itself. For that purpose, the concentration of Tapentadol is preferably at least 10 mg/mL, based on the total volume of the composition.

WO 2014/191710 discloses the opioid analgesic (R)-dihydroethorphine for use in providing local anesthesia, such as regional, epidural and spinal anesthesia. The document dies not mention Tapentadol.

WO 2016/156147 relates to an aqueous pharmaceutical composition for parenteral administration comprising Tapentadol or a physiologically acceptable salt thereof, wherein the concentration of Tapentadol is within the range of from 0.10 to 8.00 mg/mL, based on the weight of Tapentadol free base and based on the total volume of the composition; and wherein the pH value of the composition is buffered and within the range of from 4.0 to 6.0. In a preferred embodiment, the composition is adapted for local administration. The composition is adapted for parenteral administration, preferably by injection or infusion. The administration of the composition may proceed intramuscularly, intravenously, subcutaneously, epidurally, intrathecally, intraspinally and/or intracerebroventricularly. The invention disclosed in WO 2016/156147 also relates to a container comprising the pharmaceutical composition and a process for the preparation thereof. The invention disclosed in WO 2016/156147 also relates to a kit comprising the contained according to the invention in a packaging. WO 2016/156147 relates to analgesia but is fully silent on anesthesia, let alone local anesthesia.

It has been found, however, that in solutions the dissolved Tapentadol may tend to decomposition (chemical degradation) having a negative impact on storage stability of parenteral formulations.

It is an object of the invention to provide formulations of local anesthetics that have advantages compared to the formulations of local anesthetics of the prior art.

This object has been achieved by the subject-matter disclosed herein.

The inventors have unexpectedly found that Tapentadol is not only useful as analgesic, but also as local anesthetic.

Further, the inventors have unexpectedly found that chemical stability of Tapentadol can be substantially improved by providing an adjusted and robustly maintained pH value. While conventional diluted solutions of Tapentadol are instable and show a successive increase of the pH value after autoclaving and long-term storage, the pH value of the pharmaceutical composition according to the invention remains substantially unchanged.

Therefore, it has been surprisingly found that adjusting, especially lowering the pH value of the pharmaceutical composition has a stabilizing effect so that decomposition (degradation) of Tapentadol can be significantly reduced or even suppressed, also under stress conditions after repeated autoclaving. It appears that under certain conditions antimicrobial effect of Tapentadol on the one hand (cf. WO 2012/119728) and chemical stability of Tapentadol on the other hand are both function of the pH value, but in opposite directions.

Still further, it has been surprisingly found that at low pH values below 3.0, the stability of Tapentadol is a function of the buffer concentration, whereas the buffer has a relative stabilizing effect (the higher the buffer concentration, the less degradation). In contrast, however, it has been surprisingly found that at higher pH values of 7.0, i.e. outside the pH range according to the invention, the stability of Tapentadol is also function of the buffer concentration, whereas the buffer has a relative destabilizing effect (the higher the buffer concentration, the more degradation). Unexpectedly, within the pH range according to the invention, Tapentadol is stable against chemical decomposition at various buffer concentrations.

Yet further, it has been surprisingly found that even at pH values of the composition within the range of from 4.0 to 6.0, stable pharmaceutical compositions can be provided that need neither a preservative nor an antioxidant and are nevertheless storage stable for a long period of time.

Furthermore, it has been surprisingly found that the composition remains chemically stable under the harsh conditions of autoclaving, e.g. for at least 20 min at 2 bar and 121° C. Thus, the storage stability of the pharmaceutical composition according to the invention does not need to rely on the antimicrobial effect of Tapentadol alone. Autoclaving achieves sufficient storage stability against antimicrobial decontamination without the need for preservatives.

A first aspect of the invention relates to a pharmaceutical composition comprising Tapentadol or a physiologically acceptable salt thereof for use in local anesthesia, i.e. as local anesthetic.

Another aspect of the invention relates to the use of Tapentadol or a physiologically acceptable salt thereof for the manufacture of a pharmaceutical composition for local anesthesia.

Still another aspect of the invention relates to a method of providing local anesthesia in a subject in need thereof comprising administering an effective amount of a pharmaceutical composition comprising Tapentadol or a physiologically acceptable salt thereof.

According to the invention, local anesthesia is defined as a state of local or regional insensitiveness, preferably for the purpose of surgery or a diagnostic measure. The inventive use of Tapentadol as local anesthetic preferably serves the purpose of preventing pain during surgery or a diagnostic measure, although insensitiveness in local anesthesia is typically not only to pain, but to various stimuli including pain stimuli, temperature stimuli, contact stimuli, and the like.

Preferably, local anesthesia according to the invention involves a technique of local anesthesia selected from the group consisting of topical anesthesia, surface anesthesia, infiltration, plexus block, epidural (extradural) block, and spinal anesthesia (subarachnoid block).

For the purpose of the specification, local anesthesia means an anesthesia that is locally or regionally limited to a part of the body of the patient. Thus, the pharmaceutical composition according to the invention is not for use in general anesthesia and hence the pharmaceutical composition according to the invention is preferably not administered systemically, e.g. parenterally (which usually serves the purpose of systemic administration).

Preferably, the pharmaceutical composition according to the invention is for use in local anesthesia
(i) during surgery, wherein surgery is preferably selected from the group consisting of
   abdominal surgery (e.g. epidural anesthesia or spinal anesthesia, in each case preferably combined with general anesthesia);
   bone and joint surgery of the pelvis, hip, and leg (e.g. spinal anesthesia, epidural anesthesia, peripheral nerve blocks, or intravenous regional anesthesia);
   dentistry (e.g. surface anesthesia, infiltration anesthesia, intraligamentary anesthesia during restorative operations or extractions, or regional nerve blocks during extractions and surgeries);
   eye surgery (e.g. surface anesthesia with topical anesthetics or retrobulbar block);
   ears nose throat (ENT) operations, head and neck surgery (e.g. infiltration anesthesia, field blocks, or peripheral nerve blocks, plexus anesthesia);
   gynecological, obstetrical, and urological operations (spinal anesthesia or epidural anesthesia);
   heart and lung surgery (e.g. epidural anesthesia, preferably combined with general anesthesia);
   shoulder and arm surgery (e.g. plexus anesthesia or intravenous regional anesthesia);
   surgery of skin and peripheral blood vessels (e.g. topical anesthesia, field blocks, peripheral nerve blocks, spinal anesthesia, or epidural anesthesia); and
   podiatry (e.g. cutaneous, nail avulsions, matricectomy, or other podiatric procedures); or
(ii) during a diagnostic measure; wherein the diagnostic measure is preferably selected from the group consisting of
   punctures (e.g. venipuncture (blood collection), placement of intravenous cannulae, ascites drainage, or amniocentesis); and
   endoscopic procedures (e.g. bronchoscopy or cystoscopy).

The term “pharmaceutical composition” according to the invention includes any pharmaceutical preparation or formulation that is customized for being administered to a human being or animal Preferably, the composition is an aqueous liquid, preferably an aqueous solution.

Unless expressly stated otherwise, all percentages are weight percent, relative to the total weight of the pharmaceutical composition according to the invention.

Unless expressly stated otherwise, all values in mL and L refer to the total volume of the pharmaceutical composition according to the invention.

Unless expressly stated otherwise, parameters and conditions (such as temperature, pressure, relative humidity, volume, weight, concentration, pH value, titration acidity, capacity of buffer system, osmolarity, storage stability, color, and the like) are determined and measured in accordance with the requirements and recommendations as set forth in the European Pharmacopoeia (Ph. Eur.). Unless expressly stated otherwise, all references to Ph. Eur. refer to the version that is officially valid in February 2017. General conditions are typically ambient conditions.

For the purpose of the specification, the term “Tapentadol” includes the free base ((1R,2R)-3-(3-dimethylamino-1-ethyl-2-methyl-propyl)-phenol) as well as any physiologically acceptable salt thereof, particularly the hydrochloride salt ((1R,2R)-3-(3-dimethylamino-1-ethyl-2-methyl-propyl)-phenol hydrochloride).

Thus, unless expressly stated otherwise, the term “Tapentadol” does not only refer to the free base but also to any physiologically acceptable salt. Further, unless expressly stated otherwise, all amounts, contents and concentrations are equivalents related to Tapentadol free base.

Preferably, Tapentadol is present in the pharmaceutical composition according to the invention as Tapentadol hydrochloride. In a preferred embodiment, Tapentadol is present as solubilized Tapentadol hydrochloride salt form A. Form A of Tapentadol hydrochloride is known from the prior art. In this regard, it can be referred to e.g. US 2007/0213405. Preferably, form A is characterized by showing at least one or more X-ray lines (2-theta values) in a powder diffraction pattern when measured using Cu Kα radiation selected from the list comprising 15.1±0.2, 16.0±0.2, 18.9±0.2, 20.4±0.2, 22.5±0.2, 27.3±0.2, 29.3±0.2 and 30.4±0.2.

The concentration of Tapentadol in the pharmaceutical composition according to the invention is not particularly limited. Preferably, the concentration of Tapentadol is at least 0.50 mg/mL, more preferably at least 1.00 mg/mL, still more preferably at least 4.00 mg/mL, yet more preferably at least 5.00 mg/mL, based on the weight of Tapentadol free base and based on the total volume of the composition.

Preferably, the concentration of Tapentadol in the pharmaceutical composition according to the invention is greater than 8.00 mg/mL, preferably at least 8.10 mg/mL, based on the weight of Tapentadol free base and based on the total volume of the composition.

Preferably, the concentration of Tapentadol in the pharmaceutical composition according to the invention is at least 8.50 mg/mL or at least 9.00 mg/mL, more preferably at least 9.50 mg/mL or at least 10.00 mg/mL, still more preferably at least 11.00 mg/mL or at least 12.00 mg/mL, yet more preferably at least 13.00 mg/mL or at least 14.00 mg/mL, even more preferably at least 15.00 mg/mL or at least 16.00 mg/mL, most preferably at least 17.00 mg/mL or at least 18.00 mg/mL, and in particular at least 19.00 mg/mL or at least 20.00 mg/mL, based on the weight of Tapentadol free base and based on the total volume of the composition.

In preferred embodiments according to the invention, the concentration of Tapentadol in the pharmaceutical composition according to the invention is at least 21 mg/mL or at least 22 mg/mL, more preferably at least 23 mg/mL or at least 24 mg/mL, still more preferably at least 25 mg/mL or at least 27.5 mg/mL, yet more preferably at least 30 mg/mL or at least 35 mg/mL, even more preferably at least 40 mg/mL or at least 45 mg/mL, most preferably at least 50 mg/mL or at least 60 mg/mL or at least 70 mg/mL, and in particular at least 80 mg/mL or at least 90 mg/mL or at least 100 mg/mL, based on the weight of Tapentadol free base and based on the total volume of the composition.

Preferably, the concentration of Tapentadol in the pharmaceutical composition according to the invention is at most 100 mg/mL or at most 97.5 mg/mL, more preferably at most 95 mg/mL or at most 92.5 mg/mL, still more preferably at most 90 mg/mL or at most 87.5 mg/mL, yet more preferably at most 85 mg/mL or at most 82.5 mg/mL, even more preferably at most 80 mg/mL or at most 77.5 mg/mL, most preferably at most 75 mg/mL or at most 72.5 mg/mL, and in particular at most 70 mg/mL or at most 67.5 mg/mL, based on the weight of Tapentadol free base and based on the total volume of the composition.

In preferred embodiments according to the invention, the concentration of Tapentadol in the pharmaceutical composition according to the invention is at most 65.00 mg/mL or at most 60.00 mg/mL, more preferably at most 57.50 mg/mL or at most 55.00 mg/mL, still more preferably at most 52.50 mg/mL or at most 50.00 mg/mL, yet more preferably at most 47.50 mg/mL or at most 45.00 mg/mL, even more preferably at most 42.50 mg/mL or at most 40.00 mg/mL, most preferably at most 37.50 mg/mL or at most 35.00 mg/mL, and in particular at most 32.50 mg/mL or at most 30.00 mg/mL, based on the weight of Tapentadol free base and based on the total volume of the composition.

According to a preferred embodiment, the concentration of Tapentadol in the pharmaceutical composition according to the invention is within the range of 10.0±1.5 mg/mL, more preferably 10.0±1.4 mg/mL, still more preferably 10.0±1.3 mg/mL, yet more preferably 10.0±1.2 mg/mL, even more preferably 10.0±1.1 mg/mL, most preferably 10.0±1.0 mg/mL, and in particular 10.0±0.9 mg/mL, based on the weight of Tapentadol free base and based on the total volume of the composition.

According to a preferred embodiment, the concentration of Tapentadol in the pharmaceutical composition according to the invention is within the range of 12.5±4.0 mg/mL, more preferably 12.5±3.5 mg/mL, still more preferably 12.5±3.0 mg/mL, yet more preferably 12.5±2.5 mg/mL, even more preferably 12.5±2.0 mg/mL, most preferably 12.5±1.5 mg/mL, and in particular 12.5±1.0 mg/mL, based on the weight of Tapentadol free base and based on the total volume of the composition.

According to a preferred embodiment, the concentration of Tapentadol in the pharmaceutical composition according to the invention is within the range of 15±6.5 mg/mL, more preferably 15±6.0 mg/mL, still more preferably 15±5.0 mg/mL, yet more preferably 15±4.0 mg/mL, even more preferably 15±3.0 mg/mL, most preferably 15±2.0 mg/mL, and in particular 15±1.9 mg/mL, based on the weight of Tapentadol free base and based on the total volume of the composition.

According to a preferred embodiment, the concentration of Tapentadol in the pharmaceutical composition according to the invention is within the range of 17.5±9.0 mg/mL, more preferably 17.5±8.0 mg/mL, still more preferably 17.5±7.0 mg/mL, yet more preferably 17.5±6.0 mg/mL, even more preferably 17.5±5.0 mg/mL, most preferably 17.5±4.0 mg/mL, and in particular 17.5±3.0 mg/mL, based on the weight of Tapentadol free base and based on the total volume of the composition.

According to a preferred embodiment, the concentration of Tapentadol in the pharmaceutical composition according to the invention is within the range of 20±11.5 mg/mL, more preferably 20±10 mg/mL, still more preferably 20±9 mg/mL, yet more preferably 20±8 mg/mL, even more preferably 20±7 mg/mL, most preferably 20±6 mg/mL, and in particular 20±5 mg/mL, based on the weight of Tapentadol free base and based on the total volume of the composition.

According to a preferred embodiment, the concentration of Tapentadol in the pharmaceutical composition according to the invention is within the range of 25±16.5 mg/mL, more preferably 25±15 mg/mL, still more preferably 25±13 mg/mL, yet more preferably 25±11 mg/mL, even more preferably 25±9 mg/mL, most preferably 25±7 mg/mL, and in particular 25±5 mg/mL, based on the weight of Tapentadol free base and based on the total volume of the composition.

According to a preferred embodiment, the concentration of Tapentadol in the pharmaceutical composition according to the invention is within the range of 30±21.5 mg/mL, more preferably 30±21 mg/mL, still more preferably 30±18 mg/mL, yet more preferably 30±15 mg/mL, even more preferably 30±12 mg/mL, most preferably 30±9 mg/mL, and in particular 30±6 mg/mL, based on the weight of Tapentadol free base and based on the total volume of the composition.

According to a preferred embodiment, the concentration of Tapentadol in the pharmaceutical composition according to the invention is within the range of 40±31.5 mg/mL, more preferably 40±28 mg/mL, still more preferably 40±24 mg/mL, yet more preferably 40±20 mg/mL, even more preferably 40±16 mg/mL, most preferably 40±12 mg/mL, and in particular 40±8 mg/mL, based on the weight of Tapentadol free base and based on the total volume of the composition.

According to a preferred embodiment, the concentration of Tapentadol in the pharmaceutical composition according to the invention is within the range of 50±41.5 mg/mL, more preferably 50±40 mg/mL, still more preferably 50±35 mg/mL, yet more preferably 50±30 mg/mL, even more preferably 50±25 mg/mL, most preferably 50±20 mg/mL, and in particular 50±15 mg/mL, based on the weight of Tapentadol free base and based on the total volume of the composition.

According to a preferred embodiment, the concentration of Tapentadol in the pharmaceutical composition according to the invention is within the range of 60±51.5 mg/mL, more preferably 60±48 mg/mL, still more preferably 60±42 mg/mL, yet more preferably 60±36 mg/mL, even more preferably 60±30 mg/mL, most preferably 60±24 mg/mL, and in particular 60±18 mg/mL, based on the weight of Tapentadol free base and based on the total volume of the composition.

In a preferred embodiment, Tapentadol or a physiologically acceptable salt thereof is the only pharmacologically active ingredient that is contained in the pharmaceutical composition according to the invention.

In another preferred embodiment, the pharmaceutical composition according to the invention comprises Tapentadol or a physiologically acceptable salt thereof in combination with another pharmacologically active ingredient, wherein said another pharmacologically active ingredient is preferably selected from
local anesthetics (e.g. amethocaine, bupivacaine, dibucaine, levobupivacaine, lidocaine, mepivacaine, prilocaine, procaine, or ropivacaine; but preferably not lidocaine); and
vasoconstrictors (e.g. adrenalin, noradrenalin, or phenylephrine).

The pharmaceutical composition according to the invention is preferably an aqueous composition, preferably and aqueous liquid, i.e. the composition preferably comprises water which is typically water for injection purposes, i.e. highly pure and sterile water.

Preferably, the water content is at least 50 wt.-%, more preferably at least 60 wt.-%, still more preferably at least 70 wt.-%, yet more preferably at least 80 wt.-%, most preferably at least 85 wt.-% and in particular at least 90 wt.-%, based on the total weight of the composition.

Preferably, the water content is at least 95 wt.-%, more preferably at least 96 wt.-%, still more preferably at least 97 wt.-%, yet more preferably at least 98 wt.-%, most preferably at least 99 wt.-% and in particular at least 99.5 wt.-%, based on the total weight of the composition.

Besides water, the pharmaceutical composition according to the invention may contain further solvents.

Further suitable solvents include all types of physiologically acceptable hydrophilic solvents, preferably selected from the group consisting of ethanol, glycerol, propylene glycol, 1,3-butanediol and macrogol 300.

Preferably, however, the pharmaceutical composition according to the invention does not contain further solvents besides water.

The pH value of the pharmaceutical composition according to the invention is preferably buffered, i.e. the composition comprises a buffer system (i.e. a pair of at least one conjugate acid and at least one conjugate base).

Preferably, the pharmaceutical composition according to the invention comprises a buffer system comprising at least one conjugate base and at least one conjugate acid, wherein said at least one conjugate base and said at least one conjugate acid independently of one another comprise one or more protonated or deprotonated acidic functional groups independently of one another selected from the group consisting of carboxylate (—C(═O)OH), sulfate (—OS(═O)₂OH), sulfonate (—S(═O)₂OH), phosphate (—OP(═O)(OH)₂), and phosphonate (—P(═O)(OH)₂). Carboxylate is the most preferred acidic functional group (protonated: —C(═O)OH, deprotonated —C(═O)O⁻).

Preferred buffer systems are derived from the following acids: organic acids such as acetic acid, propionic acid, maleic acid, fumaric acid, lactic acid, malonic acid, malic acid, mandelic acid, citric acid, tartric acid, succinic acid; or inorganic acids such as phosphoric acid.

When the buffer system is derived from any of the above acids, the buffer system constitutes of said acid and its conjugate base(s). Buffer systems derived from acetic acid, citric acid, lactic acid, succinic acid or phosphoric acid are particularly preferred.

A skilled person is fully aware that multiprotonic acids can form more than a single pair of a conjugate acid and a conjugate base. For example, citric acid is a triprotonic acid so that it forms the following pairs of conjugate acid and conjugate base: (i) citric acid—dihydrogencitrate, (ii) dihydrogencitrate—hydrogencitrate, (iii) and hydrogencitrate—citrate. In other words, any of citric acid, dihydrogencitrate and hydrogencitrate can be the acid of a buffer system with the conjugate base. A skilled person is also fully aware that the conjugate acids and conjugate bases are in equilibrium with one another and that the predominant species that are present in a mixture of citrate, hydrogencitrate, dihydrogencitrate and citric acid can be determined on the basis of the pK_{A} values and the pH value of the composition.

For the purpose of the specification, the expression “buffer system” refers to the total quantity of conjugate acids and conjugate bases. For example, when the buffer system is derived from citric acid, i.e. is a citrate buffer system, the expression “buffer system” refers to the total quantity of citrate, hydrogencitrate, dihydrogencitrate and citric acid. Further, a skilled person is fully aware that a buffer system, e.g. citric acid as conjugate acid and sodium dihydrogencitrate as conjugate base, can be established either by adding citric acid and an appropriate amount of sodium hydroxide, or sodium citrate and an appropriate amount of hydrochloric acid, or citric acid and sodium dihydrogencitrate as such. Unless expressly stated otherwise, “sodium citrate” is synonymous to “trisodium citrate”. Sodium citrate dihydrate (=trisodium citrate dihydrate) thus has the linear formula HOC(COONa) (CH₂COONa)₂.2H₂O and a relative molecular weight of 294.10 g/mol.

Accordingly, in case that the pharmaceutical composition contains an appropriate amount of Tapentadol in form of its hydrochloride, a buffer system can be established by adding sodium citrate, e.g. in form of sodium citrate dihydrate. Nevertheless, Tapentadol and its physiologically acceptable salts are not to be considered as conjugate acid or conjugate base of the buffer system.

Preferably, the concentration of the buffer system, preferably sodium citrate or its dihydrate, is adjusted to provide a sufficient buffer system capacity.

Preferably, the pharmaceutical composition according to the invention comprises a buffer system comprising at least one conjugate base and at least one conjugate acid selected from the group consisting of citrate, hydrogencitrate, dihydrogencitrate and citric acid.

In preferred embodiments, the pharmaceutical composition according to the invention comprises a buffer system comprising at least one conjugate base and at least one conjugate acid and having a total concentration (i.e. an overall concentration of all conjugate bases and all conjugate acids of the buffer system) of at least 0.03 wt.-%, or at least 0.04 wt.-%, or at least 0.05 wt.-%, or at least 0.06 wt.-%, or at least 0.07 wt.-%, or at least 0.08 wt.-%, or at least 0.09 wt.-%, or at least 0.10 wt.-%; more preferably at least 0.11 wt.-%, or at least 0.12 wt.-%, or at least 0.13 wt.-%, or at least 0.14 wt.-%, or at least 0.15 wt.-%; still more preferably at least 0.16 wt.-%, or at least 0.17 wt.-%, or at least 0.18 wt.-%, or at least 0.19 wt.-%, or at least 0.20 wt.-%; yet more preferably at least 0.21 wt.-%, or at least 0.22 wt.-%, or at least 0.23 wt.-%, or at least 0.24 wt.-%, or at least 0.25 wt.-%; even more preferably at least 0.26 wt.-%, or at least 0.27 wt.-%, or at least 0.28 wt.-%, or at least 0.29 wt.-%, or at least 0.30 wt.-%; most preferably at least 0.35 wt.-%, or at least 0.40 wt.-%, or at least 0.45 wt.-%, or at least 0.50 wt.-%, or at least 0.55 wt.-%; and in particular at least 0.60 wt.-%, or at least 0.65 wt.-%, or at least 0.70 wt.-%, or at least 0.75 wt.-%, or at least 0.80 wt.-%, or at least 0.85 wt.-%, or at least 0.90 wt.-%, or at least 0.95 wt.-%, or at least 1.00 wt.-%, or at least 1.50 wt.-%, or at least 2.00 wt.-%, or at least 2.50 wt.-%, or at least 3.00 wt.-%, or at least 3.50 wt.-%, or at least 4.00 wt.-%, or at least 4.50 wt.-%, or at least 5.00 wt.-%; based on the total weight of the at least one conjugate base and the at least one conjugate acid and based on the total weight of the composition.

Preferably, the total concentration of said at least one conjugate base and said at least one conjugate acid is at least 0.03 wt.-%, more preferably at least 0.08 wt.-%, still more preferably at least 0.13 wt.-%, yet more preferably at least 0.18 wt.-%, and in particular at least 0.23 wt.-%, based on the total weight of the at least one conjugate base and the at least one conjugate acid and based on the total weight of the composition.

In preferred embodiments, the pharmaceutical composition according to the invention comprises a buffer system comprising at least one conjugate base and at least one conjugate acid and having a total concentration of at most 5.0 wt.-%, or at most 4.5 wt.-%, or at most 4.0 wt.-%, or at most 3.5 wt.-%, or at most 3.0 wt.-%, or at most 2.5 wt.-%, or at most 2.0 wt.-%, or at most 1.5 wt.-%, or at most 1.45 wt.-%, or at most 1.40 wt.-%, or at most 1.35 wt.-%, or at most 1.30 wt.-%; more preferably at most 1.25 wt.-%, or at most 1.20 wt.-%, or at most 1.15 wt.-%, or at most 1.20 wt.-%, or at most 1.15 wt.-%; still more preferably at most 1.10 wt.-%, or at most 1.05 wt.-%, or at most 1.00 wt.-%, or at most 0.95 wt.-%, or at most 0.90 wt.-%, yet more preferably at most 0.85 wt.-%, or at most 0.80 wt.-%, or at most 0.75 wt.-%, or at most 0.70 wt.-%, or at most 0.65 wt.-%; even more preferably at most 0.60 wt.-%, or at most 0.55 wt.-%, or at most 0.50 wt.-%, or at most 0.49 wt.-%, or at most 0.48 wt.-%; most preferably at most 0.47 wt.-%, or at most 0.45 wt.-%, or at most 0.45 wt.-%, or at most 0.44 wt.-%, or at most 0.43 wt.-%; and in particular at most 0.42 wt.-%, or at most 0.41 wt.-%, or at most 0.40 wt.-%, or at most 0.39 wt.-%, or at most 0.38 wt.-%, or at most 0.37 wt.-%, or at most 0.36 wt.-%, or at most 0.35 wt.-%, or at most 0.34 wt.-%, or at most 0.33 wt.-%, or at most 0.32 wt.-%, or at most 0.31 wt.-%, or at most 0.30 wt.-%, or at most 0.29 wt.-%, or at most 0.28 wt.-%, or at most 0.27 wt.-%, or at most 0.26 wt.-%, or at most 0.25 wt.-%, or at most 0.24 wt.-%, or at most 0.23 wt.-%, or at most 0.22 wt.-%, or at most 0.21 wt.-%, or at most 0.20 wt.-%, or at most 0.19 wt.-%, or at most 0.18 wt.-%, or at most 0.17 wt.-%, or at most 0.16 wt.-%, or at most 0.15 wt.-%, or at most 0.14 wt.-%, or at most 0.13 wt.-%, or at most 0.12 wt.-%, or at most 0.11 wt.-%, or at most 0.10 wt.-%; based on the total weight of the at least one conjugate base and the at least one conjugate acid and based on the total weight of the composition.

Preferably, the total concentration of said at least one conjugate base and said at least one conjugate acid is not more than 1.16 wt.-%, more preferably not more than 1.03 wt.-%, still more preferably not more than 0.90 wt.-%, yet more preferably not more than 0.77 wt.-%, and most preferably not more than 0.65 wt.-%, based on the total weight of the at least one conjugate base and the at least one conjugate acid and based on the total weight of the composition.

In preferred embodiments, the pharmaceutical composition according to the invention comprises a buffer system comprising at least one conjugate base and at least one conjugate acid and having a total concentration within the range of from 0.001 to 5.00 wt.-%, or from 0.001 to 4.00 wt.-%, or from 0.001 to 3.00 wt.-%, or from 0.001 to 2.00 wt.-%, or from 0.001 to 1.00 wt.-%, more preferably within the range of from 0.005 to 0.90 wt.-%, still more preferably within the range of from 0.010 to 0.80 wt.-%, yet more preferably within the range of from 0.015 to 0.70 wt.-%, even more preferably within the range of from 0.020 to 0.65 wt.-%, most preferably within the range of from 0.025 to 0.60 wt.-%, and in particular within the range of from 0.030 to 0.55 wt.-% based on the total weight of the at least one conjugate base and the at least one conjugate acid and based on the total weight of the composition.

Preferably, the total concentration of said at least one conjugate base and said at least one conjugate acid is within the range of from 0.03 to 1.16 wt.-%, more preferably within the range of from 0.08 to 1.03 wt.-%, still more preferably within the range of from 0.13 to 0.90 wt.-%, and most preferably within the range of from 0.18 to 0.77 wt.-%, based on the total weight of the at least one conjugate base and the at least one conjugate acid and based on the total weight of the composition.

Preferably, the buffers system comprises sodium citrate or its dihydrate such that depending upon the adjusted pH value of the composition, citrate, hydrogencitrate, dihydrogencitrate and citric acid are in equilibrium with one another.

In a preferred embodiment, the content of the buffer system, preferably sodium citrate or its dihydrate, is within the range of from 0.020±0.018 wt.-%, or 0.020±0.016 wt.-%, or 0.020±0.014 wt.-%, or 0.020±0.012 wt.-%, or 0.020±0.010 wt.-%, or 0.020±0.008 wt.-%, or 0.020±0.006 wt.-%, or 0.020±0.004 wt.-%, based on the total weight of the at least one conjugate base and the at least one conjugate acid and based on the total weight of the composition.

In another preferred embodiment, the content of the buffer system, preferably sodium citrate or its dihydrate, is within the range of from 0.030±0.018 wt.-%, or 0.030±0.016 wt.-%, or 0.030±0.014 wt.-%, or 0.030±0.012 wt.-%, or 0.030±0.010 wt.-%, or 0.030±0.008 wt.-%, or 0.030±0.006 wt.-%, or 0.030±0.004 wt.-%, based on the total weight of the at least one conjugate base and the at least one conjugate acid and based on the total weight of the composition.

In still another preferred embodiment, the content of the buffer system, preferably sodium citrate or its dihydrate, is within the range of from 0.040±0.035 wt.-%, or 0.040±0.030 wt.-%, or 0.040±0.025 wt.-%, or 0.040±0.020 wt.-%, or 0.040±0.015 wt.-%, or 0.040±0.010 wt.-%, or 0.040±0.005 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.050±0.035 wt.-%, or 0.050±0.030 wt.-%, or 0.050±0.025 wt.-%, or 0.050±0.020 wt.-%, or 0.050±0.015 wt.-%, or 0.050±0.010 wt.-%, or 0.050±0.005 wt.-%, based on the total weight of the at least one conjugate base and the at least one conjugate acid and based on the total weight of the composition.

In even another preferred embodiment, the content of the buffer system, preferably sodium citrate or its dihydrate, is within the range of from 0.060±0.035 wt.-%, or 0.060±0.030 wt.-%, or 0.060±0.025 wt.-%, or 0.060±0.020 wt.-%, or 0.060±0.015 wt.-%, or 0.060±0.010 wt.-%, or 0.060±0.005 wt.-%, based on the total weight of the at least one conjugate base and the at least one conjugate acid and based on the total weight of the composition.

In a further preferred embodiment, the content of the buffer system, preferably sodium citrate or its dihydrate, is within the range of from 0.070±0.035 wt.-%, or 0.070±0.030 wt.-%, or 0.070±0.025 wt.-%, or 0.070±0.020 wt.-%, or 0.070±0.015 wt.-%, or 0.070±0.010 wt.-%, or 0.070±0.005 wt.-%, based on the total weight of the at least one conjugate base and the at least one conjugate acid and based on the total weight of the composition.

In another preferred embodiment, the content of the buffer system, preferably sodium citrate or its dihydrate, is within the range of from 0.080±0.035 wt.-%, or 0.080±0.030 wt.-%, or 0.080±0.025 wt.-%, or 0.080±0.020 wt.-%, or 0.080±0.015 wt.-%, or 0.080±0.010 wt.-%, or 0.080±0.005 wt.-%, based on the total weight of the at least one conjugate base and the at least one conjugate acid and based on the total weight of the composition.

In still another preferred embodiment, the content of the buffer system, preferably sodium citrate or its dihydrate, is within the range of from 0.090±0.035 wt.-%, or 0.090±0.030 wt.-%, or 0.090±0.025 wt.-%, or 0.090±0.020 wt.-%, or 0.090±0.015 wt.-%, or 0.090±0.010 wt.-%, or 0.090±0.005 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.100±0.070 wt.-%, or 0.100±0.060 wt.-%, or 0.100±0.050 wt.-%, or 0.100±0.040 wt.-%, or 0.100±0.030 wt.-%, or 0.100±0.020 wt.-%, or 0.100±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.120±0.070 wt.-%, or 0.120±0.060 wt.-%, or 0.120±0.050 wt.-%, or 0.120±0.040 wt.-%, or 0.120±0.030 wt.-%, or 0.120±0.020 wt.-%, or 0.120±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.140±0.070 wt.-%, or 0.140±0.060 wt.-%, or 0.140±0.050 wt.-%, or 0.140±0.040 wt.-%, or 0.140±0.030 wt.-%, or 0.140±0.020 wt.-%, or 0.140±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.160±0.070 wt.-%, or 0.160±0.060 wt.-%, or 0.160±0.050 wt.-%, or 0.160±0.040 wt.-%, or 0.160±0.030 wt.-%, or 0.160±0.020 wt.-%, or 0.160±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.180±0.070 wt.-%, or 0.180±0.060 wt.-%, or 0.180±0.050 wt.-%, or 0.180±0.040 wt.-%, or 0.180±0.030 wt.-%, or 0.180±0.020 wt.-%, or 0.180±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.200±0.070 wt.-%, or 0.200±0.060 wt.-%, or 0.200±0.050 wt.-%, or 0.200±0.040 wt.-%, or 0.200±0.030 wt.-%, or 0.200±0.020 wt.-%, or 0.200±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.220±0.070 wt.-%, or 0.220±0.060 wt.-%, or 0.220±0.050 wt.-%, or 0.220±0.040 wt.-%, or 0.220±0.030 wt.-%, or 0.220±0.020 wt.-%, or 0.220±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.240±0.070 wt.-%, or 0.240±0.060 wt.-%, or 0.240±0.050 wt.-%, or 0.240±0.040 wt.-%, or 0.240±0.030 wt.-%, or 0.240±0.020 wt.-%, or 0.240±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.260±0.070 wt.-%, or 0.260±0.060 wt.-%, or 0.260±0.050 wt.-%, or 0.260±0.040 wt.-%, or 0.260±0.030 wt.-%, or 0.260±0.020 wt.-%, or 0.260±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.280±0.070 wt.-%, or 0.280±0.060 wt.-%, or 0.280±0.050 wt.-%, or 0.280±0.040 wt.-%, or 0.280±0.030 wt.-%, or 0.280±0.020 wt.-%, or 0.280±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.300±0.070 wt.-%, or 0.300±0.060 wt.-%, or 0.300±0.050 wt.-%, or 0.300±0.040 wt.-%, or 0.300±0.030 wt.-%, or 0.300±0.020 wt.-%, or 0.300±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.350±0.070 wt.-%, or 0.350±0.060 wt.-%, or 0.350±0.050 wt.-%, or 0.350±0.040 wt.-%, or 0.350±0.030 wt.-%, or 0.350±0.020 wt.-%, or 0.350±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.400±0.070 wt.-%, or 0.400±0.060 wt.-%, or 0.400±0.050 wt.-%, or 0.400±0.040 wt.-%, or 0.400±0.030 wt.-%, or 0.400±0.020 wt.-%, or 0.400±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.450±0.070 wt.-%, or 0.450±0.060 wt.-%, or 0.450±0.050 wt.-%, or 0.450±0.040 wt.-%, or 0.450±0.030 wt.-%, or 0.450±0.020 wt.-%, or 0.450±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.500±0.070 wt.-%, or 0.500±0.060 wt.-%, or 0.500±0.050 wt.-%, or 0.500±0.040 wt.-%, or 0.500±0.030 wt.-%, or 0.500±0.020 wt.-%, or 0.500±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.550±0.070 wt.-%, or 0.550±0.060 wt.-%, or 0.550±0.050 wt.-%, or 0.550±0.040 wt.-%, or 0.550±0.030 wt.-%, or 0.550±0.020 wt.-%, or 0.550±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.600±0.070 wt.-%, or 0.600±0.060 wt.-%, or 0.600±0.050 wt.-%, or 0.600±0.040 wt.-%, or 0.600±0.030 wt.-%, or 0.600±0.020 wt.-%, or 0.600±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.650±0.070 wt.-%, or 0.650±0.060 wt.-%, or 0.650±0.050 wt.-%, or 0.650±0.040 wt.-%, or 0.650±0.030 wt.-%, or 0.650±0.020 wt.-%, or 0.650±0.010 wt.-%, based on the total weight of the at least one conjugate base and the at least one conjugate acid and based on the total weight of the composition.

In yet another preferred embodiment, the content of the buffer system, preferably sodium citrate or its dihydrate, is within the range of from 0.700±0.070 wt.-%, or 0.700±0.060 wt.-%, or 0.700±0.050 wt.-%, or 0.700±0.040 wt.-%, or 0.700±0.030 wt.-%, or 0.700±0.020 wt.-%, or 0.700±0.010 wt.-%, based on the total weight of the at least one conjugate base and the at least one conjugate acid and based on the total weight of the composition.

When the buffer system is neither sodium citrate nor its dihydrate, but a different buffer system, the content of said different buffer system preferably amounts to an equivalent content that is necessary to achieve the same buffer system capacity at the given pH value as if the buffer system would be sodium citrate or its dihydrate in the above content in wt.-%.

In preferred embodiments, the pharmaceutical composition according to the invention comprises a buffer system comprising at least one conjugate base and at least one conjugate acid and having a total concentration of at least 0.1 mmol/L, or at least 0.2 mmol/L, or at least 0.3 mmol/L, or at least 0.4 mmol/L, or at least 0.5 mmol/L; more preferably at least 0.6 mmol/L, or at least 0.7 mmol/L, or at least 0.8 mmol/L, or at least 0.9 mmol/L, or at least 1.0 mmol/L; still more preferably at least 1.2 mmol/L, or at least 1.4 mmol/L, or at least 1.6 mmol/L, or at least 1.8 mmol/L, or at least 2.0 mmol/L; yet more preferably at least 2.2 mmol/L, or at least 2.4 mmol/L, or at least 2.6 mmol/L, or at least 2.8 mmol/L, or at least 3.0 mmol/L; even more preferably at least 3.2 mmol/L, or at least 3.4 mmol/L, or at least 3.6 mmol/L, or at least 3.8 mmol/L, or at least 4.0 mmol/L; most preferably at least 4.2 mmol/L, or at least 4.4 mmol/L, or at least 4.6 mmol/L, or at least 4.8 mmol/L, or at least 5.0 mmol/L; and in particular at least 5.2 mmol/L, or at least 5.4 mmol/L, or at least 5.6 mmol/L, or at least 5.8 mmol/L, or at least 6.0 mmol/L; based on the total content of the at least one conjugate base and the at least one conjugate acid and based on the total volume of the composition.

Preferably, the total concentration of said at least one conjugate base and said at least one conjugate acid is at least 1.0 mmol/L, more preferably at least 3.0 mmol/L, still more preferably at least 5.0 mmol/L, yet more preferably at least 7.0 mmol/L, and most preferably at least 9.0 mmol/L, based on the total content of the at least one conjugate base and the at least one conjugate acid and based on the total volume of the composition.

In preferred embodiments, the pharmaceutical composition according to the invention comprises a buffer system comprising at least one conjugate base and at least one conjugate acid, wherein said at least one conjugate base and said at least one conjugate acid independently of one another comprise one or more protonated or deprotonated acidic functional groups independently of one another selected from the group consisting of carboxylate, sulfate, sulfonate, phosphate, and phosphonate; wherein the total concentration of said protonated or deprotonated acidic functional groups (equivalents) is at least 0.3 mmol-eq/L, or at least 0.6 mmol-eq/L, or at least 0.9 mmol-eq/L, or at least 1.2 mmol-eq/L, or at least 1.5 mmol-eq/L; more preferably at least 1.8 mmol-eq/L, or at least 2.1 mmol-eq/L, or at least 2.4 mmol-eq/L, or at least 2.7 mmol-eq/L, or at least 3.0 mmol-eq/L; still more preferably at least 3.6 mmol-eq/L, or at least 4.2 mmol-eq/L, or at least 4.8 mmol-eq/L, or at least 5.4 mmol-eq/L, or at least 6.0 mmol-eq/L; yet more preferably at least 6.6 mmol-eq/L, or at least 7.2 mmol-eq/L, or at least 7.8 mmol-eq/L, or at least 8.4 mmol-eq/L, or at least 9.0 mmol-eq/L; even more preferably at least 9.6 mmol-eq/L, or at least 10.2 mmol-eq/L, or at least 10.8 mmol-eq/L, or at least 11.4 mmol-eq/L, or at least 12.0 mmol-eq/L; most preferably at least 12.6 mmol-eq/L, or at least 13.2 mmol-eq/L, or at least 13.8 mmol-eq/L, or at least 14.4 mmol-eq/L, or at least 15.0 mmol-eq/L; and in particular at least 15.6 mmol-eq/L, or at least 16.2 mmol-eq/L, or at least 16.8 mmol-eq/L, or at least 17.4 mmol-eq/L, or at least 18.0 mmol-eq/L; based on the total quantity of said protonated or deprotonated acidic functional groups and based on the total volume of the composition.

In preferred embodiments, the pharmaceutical composition according to the invention comprises a buffer system comprising at least one conjugate base and at least one conjugate acid and having a total concentration of at most 100 mmol/L, or at most 95 mmol/L, or at most 90 mmol/L, or at most 85 mmol/L, or at most 80 mmol/L; more preferably at most 78 mmol/L, or at most 76 mmol/L, or at most 74 mmol/L, or at most 72 mmol/L, or at most 70 mmol/L; still more preferably at most 68 mmol/L, or at most 66 mmol/L, or at most 64 mmol/L, or at most 62 mmol/L, or at most 60 mmol/L; yet more preferably at most 58 mmol/L, or at most 56 mmol/L, or at most 54 mmol/L, or at most 52 mmol/L, or at most 50 mmol/L; even more preferably at most 48 mmol/L, or at most 46 mmol/L, or at most 44 mmol/L, or at most 42 mmol/L, or at most 40 mmol/L; most preferably at most 38 mmol/L, or at most 36 mmol/L, or at most 34 mmol/L, or at most 32 mmol/L, or at most 30 mmol/L; and in particular at most 28 mmol/L, or at most 26 mmol/L, or at most 24 mmol/L, or at most 22 mmol/L, or at most 20 mmol/L; based on the total content of the at most one conjugate base and the at most one conjugate acid and based on the total volume of the composition.

Preferably, the total concentration of said at least one conjugate base and said at least one conjugate acid is not more than 200 mmol/L, more preferably not more than 150 mmol/L, still more preferably not more than 100 mmol/L, yet more preferably not more than 75 mmol/L, and most preferably not more than 50 mmol/L, based on the total content of the at least one conjugate base and the at least one conjugate acid and based on the total volume of the composition.

Preferably, the total concentration of said at least one conjugate base and said at least one conjugate acid is not more than 45 mmol/L, more preferably not more than 40 mmol/L, still more preferably not more than 35 mmol/L, yet more preferably not more than 30 mmol/L, and most preferably not more than 25 mmol/L, based on the total content of the at least one conjugate base and the at least one conjugate acid and based on the total volume of the composition.

In preferred embodiments, the pharmaceutical composition according to the invention comprises a buffer system comprising at least one conjugate base and at least one conjugate acid, wherein said at least one conjugate base and said at least one conjugate acid independently of one another comprise one or more protonated or deprotonated acidic functional groups independently of one another selected from the group consisting of carboxylate, sulfate, sulfonate, phosphate, and phosphonate; wherein the total concentration of said protonated or deprotonated acidic functional groups (equivalents) is at most 300 mmol-eq/L, or at most 285 mmol-eq/L, or at most 270 mmol-eq/L, or at most 255 mmol-eq/L, or at most 240 mmol-eq/L; more preferably at most 234 mmol-eq/L, or at most 228 mmol-eq/L, or at most 222 mmol-eq/L, or at most 216 mmol-eq/L, or at most 210 mmol-eq/L; still more preferably at most 204 mmol-eq/L, or at most 198 mmol-eq/L, or at most 192 mmol-eq/L, or at most 186 mmol-eq/L, or at most 180 mmol-eq/L; yet more preferably at most 174 mmol-eq/L, or at most 168 mmol-eq/L, or at most 162 mmol-eq/L, or at most 156 mmol-eq/L, or at most 150 mmol-eq/L; even more preferably at most 144 mmol-eq/L, or at most 138 mmol-eq/L, or at most 132 mmol-eq/L, or at most 126 mmol-eq/L, or at most 120 mmol-eq/L; most preferably at most 114 mmol-eq/L, or at most 108 mmol-eq/L, or at most 102 mmol-eq/L, or at most 96 mmol-eq/L, or at most 90 mmol-eq/L; and in particular at most 84 mmol-eq/L, or at most 78 mmol-eq/L, or at most 72 mmol-eq/L, or at most 66 mmol-eq/L, or at most 60 mmol-eq/L; based on the total quantity of said protonated or deprotonated acidic functional groups and based on the total volume of the composition.

In preferred embodiments, the pharmaceutical composition according to the invention comprises a buffer system comprising at least one conjugate base and at least one conjugate acid and having a total concentration within the range of 1.0±0.9 mmol/L, or 1.0±0.8 mmol/L, or 1.0±0.7 mmol/L, or 1.0±0.6 mmol/L, or 1.0±0.5 mmol/L, or 1.0±0.4 mmol/L, or 1.0±0.3 mmol/L; or 1.5±0.9 mmol/L, or 1.5±0.8 mmol/L, or 1.5±0.7 mmol/L, or 1.5±0.6 mmol/L, or 1.5±0.5 mmol/L, or 1.5±0.4 mmol/L, or 1.5±0.3 mmol/L; or 2.0±0.9 mmol/L, or 2.0±0.8 mmol/L, or 2.0±0.7 mmol/L, or 2.0±0.6 mmol/L, or 2.0±0.5 mmol/L, or 2.0±0.4 mmol/L, or 2.0±0.3 mmol/L; or 2.5±0.9 mmol/L, or 2.5±0.8 mmol/L, or 2.5±0.7 mmol/L, or 2.5±0.6 mmol/L, or 2.5±0.5 mmol/L, or 2.5±0.4 mmol/L, or 2.5±0.3 mmol/L; or 3.0±0.9 mmol/L, or 3.0±0.8 mmol/L, or 3.0±0.7 mmol/L, or 3.0±0.6 mmol/L, or 3.0±0.5 mmol/L, or 3.0±0.4 mmol/L, or 3.0±0.3 mmol/L; or 3.5±0.9 mmol/L, or 3.5±0.8 mmol/L, or 3.5±0.7 mmol/L, or 3.5±0.6 mmol/L, or 3.5±0.5 mmol/L, or 3.5±0.4 mmol/L, or 3.5±0.3 mmol/L; or 4.0±0.9 mmol/L, or 4.0±0.8 mmol/L, or 4.0±0.7 mmol/L, or 4.0±0.6 mmol/L, or 4.0±0.5 mmol/L, or 4.0±0.4 mmol/L, or 4.0±0.3 mmol/L; or 4.5±0.9 mmol/L, or 4.5±0.8 mmol/L, or 4.5±0.7 mmol/L, or 4.5±0.6 mmol/L, or 4.5±0.5 mmol/L, or 4.5±0.4 mmol/L, or 4.5±0.3 mmol/L; or 5.0±0.9 mmol/L, or 5.0±0.8 mmol/L, or 5.0±0.7 mmol/L, or 5.0±0.6 mmol/L, or 5.0±0.5 mmol/L, or 5.0±0.4 mmol/L, or 5.0±0.3 mmol/L; or 7.5±5.0 mmol/L, or 7.5±4.5 mmol/L, or 7.5±4.0 mmol/L, or 7.5±3.5 mmol/L, or 7.5±3.0 mmol/L, or 7.5±2.5 mmol/L, or 7.5±2.0 mmol/L, or 7.5±1.5 mmol/L, or 7.5±1.0 mmol/L; or 10±5.0 mmol/L, or 10±4.5 mmol/L, or 10±4.0 mmol/L, or 10±3.5 mmol/L, or 10±3.0 mmol/L, or 10±2.5 mmol/L, or 10±2.0 mmol/L, or 10±1.5 mmol/L, or 10±1.0 mmol/L; or 12.5±5.0 mmol/L, or 12.5±4.5 mmol/L, or 12.5±4.0 mmol/L, or 12.5±3.5 mmol/L, or 12.5±3.0 mmol/L, or 12.5±2.5 mmol/L, or 12.5±2.0 mmol/L, or 12.5±1.5 mmol/L, or 12.5±1.0 mmol/L; or 15±5.0 mmol/L, or 15±4.5 mmol/L, or 15±4.0 mmol/L, or 15±3.5 mmol/L, or 15±3.0 mmol/L, or 15±2.5 mmol/L, or 15±2.0 mmol/L, or 15±1.5 mmol/L, or 15±1.0 mmol/L; or 17.5±5.0 mmol/L, or 17.5±4.5 mmol/L, or 17.5±4.0 mmol/L, or 17.5±3.5 mmol/L, or 17.5±3.0 mmol/L, or 17.5±2.5 mmol/L, or 17.5±2.0 mmol/L, or 17.5±1.5 mmol/L, or 17.5±1.0 mmol/L; or 20±5.0 mmol/L, or 20±4.5 mmol/L, or 20±4.0 mmol/L, or 20±3.5 mmol/L, or 20±3.0 mmol/L, or 20±2.5 mmol/L, or 20±2.0 mmol/L, or 20±1.5 mmol/L, or 20±1.0 mmol/L; or 22.5±5.0 mmol/L, or 22.5±4.5 mmol/L, or 22.5±4.0 mmol/L, or 22.5±3.5 mmol/L, or 22.5±3.0 mmol/L, or 22.5±2.5 mmol/L, or 22.5±2.0 mmol/L, or 22.5±1.5 mmol/L, or 22.5±1.0 mmol/L; or 25±5.0 mmol/L, or 25±4.5 mmol/L, or 25±4.0 mmol/L, or 25±3.5 mmol/L, or 25±3.0 mmol/L, or 25±2.5 mmol/L, or 25±2.0 mmol/L, or 25±1.5 mmol/L, or 25±1.0 mmol/L; based on the total content of the at most one conjugate base and the at most one conjugate acid and based on the total volume of the composition.

Preferably, the total concentration of said at least one conjugate base and said at least one conjugate acid is within the range of from 1.0 to 45 mmol/L, more preferably within the range of from 3.0 to 40 mmol/L, still more preferably within the range of from 5.0 to 35 mmol/L, yet more preferably within the range of from 7.0 to 30 mmol/L, and most preferably within the range of from 9.0 to 25 mmol/L, based on the total content of the at least one conjugate base and the at least one conjugate acid and based on the total volume of the composition.

In preferred embodiments, the pharmaceutical composition according to the invention comprises a buffer system comprising at least one conjugate base and at least one conjugate acid, wherein said at least one conjugate base and said at least one conjugate acid independently of one another comprise one or more protonated or deprotonated acidic functional groups independently of one another selected from the group consisting of carboxylate, sulfate, sulfonate, phosphate, and phosphonate; wherein the total concentration of said protonated or deprotonated acidic functional groups (equivalents) is within the range of 3.0±2.7 mmol-eq/L, or 3.0±2.4 mmol-eq/L, or 3.0±2.1 mmol-eq/L, or 3.0±1.8 mmol-eq/L, or 3.0±1.5 mmol-eq/L, or 3.0±1.2 mmol-eq/L, or 3.0±0.9 mmol-eq/L; or 4.5±2.7 mmol-eq/L, or 4.5±2.4 mmol-eq/L, or 4.5±2.1 mmol-eq/L, or 4.5±1.8 mmol-eq/L, or 4.5±1.5 mmol-eq/L, or 4.5±1.2 mmol-eq/L, or 4.5±0.9 mmol-eq/L; or 6.0±2.7 mmol-eq/L, or 6.0±2.4 mmol-eq/L, or 6.0±2.1 mmol-eq/L, or 6.0±1.8 mmol-eq/L, or 6.0±1.5 mmol-eq/L, or 6.0±1.2 mmol-eq/L, or 6.0±0.9 mmol-eq/L; or 7.5±2.7 mmol-eq/L, or 7.5±2.4 mmol-eq/L, or 7.5±2.1 mmol-eq/L, or 7.5±1.8 mmol-eq/L, or 7.5±1.5 mmol-eq/L, or 7.5±1.2 mmol-eq/L, or 7.5±0.9 mmol-eq/L; or 9.0±2.7 mmol-eq/L, or 9.0±2.4 mmol-eq/L, or 9.0±2.1 mmol-eq/L, or 9.0±1.8 mmol-eq/L, or 9.0±1.5 mmol-eq/L, or 9.0±1.2 mmol-eq/L, or 9.0±0.9 mmol-eq/L; or 10.5±2.7 mmol-eq/L, or 10.5±2.4 mmol-eq/L, or 10.5±2.1 mmol-eq/L, or 10.5±1.8 mmol-eq/L, or 10.5±1.5 mmol-eq/L, or 10.5±1.2 mmol-eq/L, or 10.5±0.9 mmol-eq/L; or 12±2.7 mmol-eq/L, or 12±2.4 mmol-eq/L, or 12±2.1 mmol-eq/L, or 12±1.8 mmol-eq/L, or 12±1.5 mmol-eq/L, or 12±1.2 mmol-eq/L, or 12±0.9 mmol-eq/L; or 13.5±2.7 mmol-eq/L, or 13.5±2.4 mmol-eq/L, or 13.5±2.1 mmol-eq/L, or 13.5±1.8 mmol-eq/L, or 13.5±1.5 mmol-eq/L, or 13.5±1.2 mmol-eq/L, or 13.5±0.9 mmol-eq/L; or 15±2.7 mmol-eq/L, or 15±2.4 mmol-eq/L, or 15±2.1 mmol-eq/L, or 15±1.8 mmol-eq/L, or 15±1.5 mmol-eq/L, or 15±1.2 mmol-eq/L, or 15±0.9 mmol-eq/L; or 22.5±15 mmol-eq/L, or 22.5±13.5 mmol-eq/L, or 22.5±12 mmol-eq/L, or 22.5±10.5 mmol-eq/L, or 22.5±9.0 mmol-eq/L, or 22.5±7.5 mmol-eq/L, or 22.5±6.0 mmol-eq/L, or 22.5±4.5 mmol-eq/L, or 22.5±3.0 mmol-eq/L; or 30±15 mmol-eq/L, or 30±13.5 mmol-eq/L, or 30±12 mmol-eq/L, or 30±10.5 mmol-eq/L, or 30±9.0 mmol-eq/L, or 30±7.5 mmol-eq/L, or 30±6.0 mmol-eq/L, or 30±4.5 mmol-eq/L, or 30±3.0 mmol-eq/L; or 37.5±15 mmol-eq/L, or 37.5±13.5 mmol-eq/L, or 37.5±12 mmol-eq/L, or 37.5±10.5 mmol-eq/L, or 37.5±9.0 mmol-eq/L, or 37.5±7.5 mmol-eq/L, or 37.5±6.0 mmol-eq/L, or 37.5±4.5 mmol-eq/L, or 37.5±3.0 mmol-eq/L; or 45±15 mmol-eq/L, or 45±13.5 mmol-eq/L, or 45±12 mmol-eq/L, or 45±10.5 mmol-eq/L, or 45±9.0 mmol-eq/L, or 45±7.5 mmol-eq/L, or 45±6.0 mmol-eq/L, or 45±4.5 mmol-eq/L, or 45±3.0 mmol-eq/L; or 52.5±15 mmol-eq/L, or 52.5±13.5 mmol-eq/L, or 52.5±12 mmol-eq/L, or 52.5±10.5 mmol-eq/L, or 52.5±9.0 mmol-eq/L, or 52.5±7.5 mmol-eq/L, or 52.5±6.0 mmol-eq/L, or 52.5±4.5 mmol-eq/L, or 52.5±3.0 mmol-eq/L; or 60±15 mmol-eq/L, or 60±13.5 mmol-eq/L, or 60±12 mmol-eq/L, or 60±10.5 mmol-eq/L, or 60±9.0 mmol-eq/L, or 60±7.5 mmol-eq/L, or 60±6.0 mmol-eq/L, or 60±4.5 mmol-eq/L, or 60±3.0 mmol-eq/L; or 67.5±15 mmol-eq/L, or 67.5±13.5 mmol-eq/L, or 67.5±12 mmol-eq/L, or 67.5±10.5 mmol-eq/L, or 67.5±9.0 mmol-eq/L, or 67.5±7.5 mmol-eq/L, or 67.5±6.0 mmol-eq/L, or 67.5±4.5 mmol-eq/L, or 67.5±3.0 mmol-eq/L; or 75±15 mmol-eq/L, or 75±13.5 mmol-eq/L, or 75±12 mmol-eq/L, or 75±10.5 mmol-eq/L, or 75±9.0 mmol-eq/L, or 75±7.5 mmol-eq/L, or 75±6.0 mmol-eq/L, or 75±4.5 mmol-eq/L, or 75±3.0 mmol-eq/L; based on the total quantity of said protonated or deprotonated acidic functional groups and based on the total volume of the composition.

The pH value of the pharmaceutical composition according to the invention is not particularly limited. Preferably, the pH value of the pharmaceutical composition according to the invention is within the range of from 2.0 to 12, more preferably within the range of from 2.0 to 10, still more preferably within the range of from 2.0 to 9.0, yet more preferably within the range of from 2.0 to 8.0, even more preferably within the range of from 2.0 to 7.5, most preferably within the range of from 2.0 to 7.0. Preferably, the pH value of the pharmaceutical composition according to the invention is within the range of from 3.0 to 12, more preferably within the range of from 3.0 to 10, still more preferably within the range of from 3.0 to 9.0, yet more preferably within the range of from 3.0 to 8.0, even more preferably within the range of from 3.0 to 7.5, most preferably within the range of from 3.0 to 7.0.

Preferably, the pH value of the pharmaceutical composition according to the invention is within the range of from greater than 3.0 to less than 6.7. According to this definition, the pH value of 3.0 is preferably not encompassed by the pH range. According to preferred embodiments according to the invention, however, a pH value of 3.0 may be encompassed. According to these embodiments, the pH value of the pharmaceutical composition according to the invention is preferably within the range of from greater than 2.0 to less than 6.7, more preferably at least 2.1, or at least 2.2, or at least 2.3, or at least 2.4, or at least 2.5, or at least 2.6, or at least 2.7, or at least 2.8, or at least 2.9, or at least 3.0.

In preferred embodiments, the pH value of the pharmaceutical composition according to the invention is not greater than 6.6 or not greater than 6.5, more preferably not greater than 6.4 or not greater than 6.3, still more preferably not greater than 6.2 or not greater than 6.1, yet more preferably not greater than 6.0 or not greater than 5.9, even more preferably not greater than 5.8 or not greater than 5.7, most preferably not greater than 5.6 or not greater than 5.5, and in particular not greater than 5.4 or not greater than 5.3.

In preferred embodiments, the pH value of the pharmaceutical composition according to the invention is at least 3.1 or at least 3.2, more preferably at least 3.3 or at least 3.4, still more preferably at least 3.5 or at least 3.6, yet more preferably at least 3.7 or at least 3.8, even more preferably at least 3.9 or at least 4.0, most preferably at least 4.1 or at least 4.2, and in particular at least 4.3 or at least 4.4.

Preferably, the pH value of the pharmaceutical composition according to the invention is within the range of from 3.0 to 6.5, or from 3.1 to 6.5, or from 3.5 to 6.5, or from 4.0 to 6.5, or from 4.5 to 6.5, or from 5.0 to 6.5.

Preferably, the pH value of the pharmaceutical composition according to the invention is within the range of from 3.0 to 6.0, or from 3.1 to 6.0, or from 3.5 to 6.0, or from 4.0 to 6.0, or from 4.5 to 6.0, or from 5.0 to 6.0.

Preferably, the pH value of the pharmaceutical composition according to the invention is within the range of from 3.0 to 5.5, or from 3.1 to 5.5, or from 3.5 to 5.5, or from 4.0 to 5.5, or from 4.5 to 5.5, or from 5.0 to 5.5.

Preferably, the pH value of the pharmaceutical composition according to the invention is within the range of from 3.0 to 5.0, or from 3.1 to 5.0, or from 3.5 to 5.0, or from 4.0 to 5.0, or from 4.5 to 5.0.

In a preferred embodiment, the composition has a pH value within the range of 2.5±0.5, more preferably 2.5±0.4, still more preferably 2.5±0.3, yet more preferably 2.5±0.2, and in particular 2.5±0.1.

In another preferred embodiment, the composition has a pH value within the range of 2.75±0.50, more preferably 2.75±0.40, still more preferably 2.75±0.30, yet more preferably 2.75±0.20, and in particular 2.75±0.10.

In still another preferred embodiment, the composition has a pH value within the range of 3.0±1.0, more preferably 3.0±0.9, still more preferably 3.0±0.8, yet more preferably 3.0±0.7, even more preferably 3.0±0.6 or 3.0±0.5, most preferably 3.0±0.4 or 3.0±0.3, and in particular 5.0±0.2 or 5.0±0.1.

In yet another preferred embodiment, the composition has a pH value within the range of 3.25±0.50, more preferably 3.25±0.40, still more preferably 3.25±0.30, yet more preferably 3.25±0.20, and in particular 3.25±0.10.

In another preferred embodiment, the composition has a pH value within the range of 3.5±0.5, more preferably 3.5±0.4, still more preferably 3.5±0.3, yet more preferably 3.5±0.2, and in particular 3.5±0.1.

In still another preferred embodiment, the composition has a pH value within the range of 3.75±0.50, more preferably 3.75±0.40, still more preferably 3.75±0.30, yet more preferably 3.75±0.20, and in particular 3.75±0.10.

In yet another preferred embodiment, the composition has a pH value within the range of 4.0±1.0, more preferably 4.0±0.9, still more preferably 4.0±0.8, yet more preferably 4.0±0.7, even more preferably 4.0±0.6 or 4.0±0.5, most preferably 4.0±0.4 or 4.0±0.3, and in particular 4.0±0.2 or 4.0±0.1.

In a preferred embodiment, the composition has a pH value within the range of 4.25±0.50, more preferably 4.25±0.40, still more preferably 4.25±0.30, yet more preferably 4.25±0.20, and in particular 4.25±0.10.

In another preferred embodiment, the composition has a pH value within the range of 4.5±0.5, more preferably 4.5±0.4, still more preferably 4.5±0.3, yet more preferably 4.5±0.2, and in particular 4.5±0.1.

In still another preferred embodiment, the composition has a pH value within the range of 4.75±0.50, more preferably 4.75±0.40, still more preferably 4.75±0.30, yet more preferably 4.75±0.20, and in particular 4.75±0.10.

In yet another preferred embodiment, the composition has a pH value within the range of 5.0±1.0, more preferably 5.0±0.9, still more preferably 5.0±0.8, yet more preferably 5.0±0.7, even more preferably 5.0±0.6 or 5.0±0.5, most preferably 5.0±0.4 or 5.0±0.3, and in particular 5.0±0.2 or 5.0±0.1.

In another preferred embodiment, the composition has a pH value within the range of 5.25±0.50, more preferably 5.25±0.40, still more preferably 5.25±0.30, yet more preferably 5.25±0.20, and in particular 5.25±0.10.

In still another preferred embodiment, the composition has a pH value within the range of 5.5±0.5, more preferably 5.5±0.4, still more preferably 5.5±0.3, yet more preferably 5.5±0.2, and in particular 5.5±0.1.

In a preferred embodiment, the composition has a pH value within the range of 4.25±0.50, more preferably 5.75±0.40, still more preferably 5.75±0.30, yet more preferably 5.75±0.20, and in particular 5.75±0.10.

The pharmaceutical composition according to the invention may be administered by any route that is suitable for providing local anesthesia.

Preferably, the pharmaceutical composition according to the invention is administered locally. Preferably, the pharmaceutical composition according to the invention is administered at a location and via a route such that it has a local anesthetizing effect on specific nerve pathways (local anesthetic nerve block).

In a preferred embodiment, the pharmaceutical composition according to the invention is administered topically, e.g. topically to the skin surface. Topical anesthesia (surface anesthesia) may involve topical administration of a pharmaceutical composition selected from the group consisting of creams, gels, ointments, liquids, or sprays. Tapentadol or a physiologically acceptable salt thereof may be dissolved in DMSO or other solvents/carriers for deeper absorption.

Preferred pharmaceutical compositions according to the invention that are preferably used for topical administration are described here below:

When the pharmaceutical composition according to the invention is for topical administration, it is preferably semisolid at room temperature. A skilled person knows how to distinguish semisolid pharmaceutical compositions from solid pharmaceutical compositions and from liquid pharmaceutical compositions. The term “semisolid” is well accepted in the art. For the purpose of the specification, the term “semisolid” preferably has the meaning as used in the Eur. Ph. Preferably, the semisolid preparation becomes liquid at a temperature of at most 100° C., more preferably of at most 95° C., still more preferably of at most 90° C., yet more preferably of at most 85° C., most preferably of at most 80° C. and in particular of at most 75° C. According to the Eur. Ph. semisolid preparations can be systematically divided into ointments (hydrophobic, hydrophilic and absorption ointments), creams (hydrophobic and hydrophilic), gels (hydrophobic and hydrophilic), pastes, poultices and plasters.

Thus, the pharmaceutical composition for topical administration is preferably selected from the group consisting of ointments, creams, gels, pastes, poultices and plasters.

Ointments are lipid-based semisolid preparations which do not necessarily contain an aqueous phase and appear homogeneous. In simplified terms, they are characterized as single-phase preparations, although liquid and/or solid particles may be dispersed therein. Typical base formulations for hydrophobic ointments contain hard paraffin, soft paraffin, vegetable oils, animal fats, hydrogenated oils, synthetic oily materials, partially synthetic glycerides and/or waxes. Hydrophobic ointments can only absorb small amounts of water. Hydrophilic ointments and absorption ointments further contain emulsifiers that increase their potency to absorb water. Hydrophilic ointments are based on formulations that are soluble in water. They often contain a polyethylene glycol.

Creams are biphasic or multiple-phase systems that contain an aqueous and lipid phase. They often have an opaque appearance as contrasted with translucent ointments. Creams contain at least one surfactant (emulsifier) whose hydrophilicity determines whether the cream is hydrophobic (e. g., w/o-type), hydrophilic (e. g., o/w-type) or amphiphilic. A cream's phase characteristics are often complex. For instance, creams may contain more phases than only the aqueous and lipid phase, e. g. an aqueous phase and two lipid phases. Or it may further contain solid particles or the lipid phase may be solid itself. Therefore, creams are usually not classified as emulsions. They may, however, be referred to as emulsoid.

Gels are semisolid systems in which a liquid is solidified by a gelling agent which forms three-dimensional crosslinked network within the liquid. The liquid may be aqueous (hydrophobic gel, oleogel) or lipid-based (hydrophilic gel, hydrogel). Gels typically exhibit no flow when in the steady-state.

Creams and ointments may also contain thickening agents and the classification is not in each and every case unambiguous. For instances, there are semisolid systems that can both be classified as o/w-creams or hydrogels, and are thus also referred to as emulsion gels.

Pastes are lipid-based preparations which contain a high amount of solid particles dispersed therein. They usually contain only a small amount of water. Preparations of this type that contain a high amount of water are usually referred to as poultices. In common parlance, free-running aqueous multiple-phase preparations that are suspensions of solid particles in hydrophilic solvents or emulsions are also referred to as lotions.

Preferably, the pharmaceutical composition for topical administration is selected from the group consisting of ointments, creams, magmas, gels, emulsions, suspensions, lotions, liniments, pastes, poultices, suspension gels and emulsion gels.

In a preferred embodiment, the pharmaceutical composition for topical administration is an emulsoid preparation such as a cream or lotion. The emulsoid preparation may be hydrophilic, hydrophobic or amphiphilic. For the purpose of specification, the term “hydrophilic emulsoid preparation” shall refer to any emulsoid preparation in which the aqueous phase represents the external phase, including biphasic preparations of the w/o-type and multiple-phase preparations of the w/o/w-type. For the purpose of specification, the term “hydrophobic emulsoid preparation” shall refer to any biphasic emulsoid preparation in which the lipid phase represents the external phase, including biphasic preparations of the w/o-type. Preferably, the emulsoid preparation is hydrophilic. In particular, the emulsoid preparation is of the o/w-type (oil-in-water) or w/o/w-type (w/o emulsoid preparation in water).

In another preferred embodiment, the pharmaceutical composition for topical administration is a suspensoid preparation, preferably selected from the group consisting of sustained release suspensions, gels and magmas, and lotions.

In a preferred embodiment, the aqueous pharmaceutical composition for topical administration is a single phase formulation. In another preferred embodiment, the pharmaceutical composition for topical administration is a two-phase or multi-phase formulation. Preferably, the pharmaceutical composition contains at least one aqueous phase and at least one lipid phase. In a preferred embodiment, the aqueous phase and the lipid phase form a bicoherent system. In another preferred embodiment, the aqueous phase and the lipid phase form an emulsoid or suspendoid system, in which they may independently of each other be present as dispersed phase and/or coherent phase. In case that the emulsoid system contains a solid phase, two or three coherent phases may be present. For example, the lipid phase may be dispersed in an aqueous phase and contain a second aqueous phase dispersed therein (w/o/w-type). Preferably, the emulsoid system contains at least one aqueous coherent phase.

Preferably, the pharmaceutical composition for topical administration contains a lipid. In a preferred embodiment, the lipid is selected from the group consisting of
saturated C₈ to C₁₈ fatty acids, such as myristic acid and stearic acid;
unsaturated C₈ to C₁₈ fatty acids and their esters, such as oleic acid and ethyl oleate;
mixtures of saturated and unsaturated C₈ to C₁₈ fatty acids, including vegetable oils such as soybean oil and peanut oil; hydrogenated oils such as hydrogenated castor oil; and animal fats; and
glycerides of fatty acids, including monoglycerides, diglycerides, triglycerides, and mixtures thereof; preferably of C₆ to C₁₂ fatty acids, more preferably of C₆ to C₁₀ fatty acids, such as the caprylic/capric triglyceride mixtures, most preferably medium-chain triglycerides according to Ph. Eur. or USP;
propylene glycol fatty acid esters such as propylene glycol monocaprylate;
waxes including wool wax, carnauba wax, bees wax and mixtures of saturated C₁₈ to C₃₂ hydrocarbons such as hard paraffin and soft paraffin (e. g. white petroleum jelly).

Preferably, the lipid is a C₈-C₁₈-fatty acid ester of a monoalcohol (e.g. C₁-C₁₂-alkylalcohols), a di-C₈-C₁₈-fatty acid ester of a dialcohol (e.g. ethylene glycol or propylene glycol) or tri-C₈-C₁₈-fatty acid ester of a trialcohol (e.g. glycerol). Especially preferred are medium-chain triglycerides according to Ph. Eur. or USP such as said caprylic/capric triglyceride mixtures.

Preferably, the pharmaceutical composition for topical administration contains a surfactant. The surfactant may act as an emulsifier, wetting agent, solubiliser and/or detergent. In a preferred embodiment, the surfactant acts as an O/W emulsifier. In another preferred embodiment, the surfactant acts as a W/O emulsifier. The surfactant may be an ionic surfactant, amphoteric surfactant or non-ionic surfactant.

In a preferred embodiment, the surfactant is ionic, in particular anionic. Suitable anionic ionic surfactants include but are not limited to sodium lauryl sulfate (sodium dodecyl sulfate), sodium cetyl stearyl sulfate, sodium dioctylsulfosuccinate (docusate sodium); and the corresponding potassium or calcium salts thereof.

In another preferred embodiment, the surfactant is cationic. Suitable cationic ionic surfactants include but are not limited to quaternary ammonium compounds, such as benzalkonium chloride, benzethonium chloride, cetylpyridinium chloride, cetyl trimethylammoniom bromide, cetyl trimethylammoniom chloride, 5-bromo-5-nitro-1,3-dioxane, dimethyldioctadecylammonium chloride and dimethyldioctadecylammonium bromide; and hydrohalide salts of amines, such as octenidine dihydrochloride.

In still another preferred embodiment, the surfactant is amphoteric. Suitable amphoteric surfactants include the group of phospholipids, e. g. lecithine.

In yet another preferred embodiment, the surfactant is non-ionic. Suitable non-ionic surfactants include but are not limited to
polyoxyethylene-sorbitan-fatty acid esters, e.g. mono- and tri-lauryl, palmityl, stearyl and oleyl esters, such as the type known under the name “polysorbat” and commercially available under the trade name “Tween®”;
polyoxyethylene-glycerol-fatty acid esters, e.g. mono- and tri-lauryl, palmityl, stearyl and oleyl esters;
polyoxyethylene fatty acid esters, the fatty acid preferably having from about 8 to about 18 carbon atoms, such as diglycol stearate, glycol stearate, glycol distearate and mixtures of polyoxyethylene esters of 12-hydroxystearic acid;
polyoxyethylene esters of alpha-tocopheryl succinate;
polyglycolyzed glycerides;
reaction products of a natural or hydrogenated castor oil and ethylene oxide;
glycerol fatty acid esters, e.g. mono- and tri-lauryl, palmityl, stearyl and oleyl esters, including diglycol stearate, glycerol monostearate, glycerol monopalmitate and glycerol trioleate;
fatty alcohols or sterols including cetyl alcohol, stearyl alcohol, cetylstearyl alcohol and cholesterol.

The pharmaceutical composition for topical administration may further contain a hydrophilic solvent. The hydrophilic solvent may be selected from the group consisting of propylene glycol, ethanol, poly(ethylene glycol) or PEG, propylene carbonate, diethylene glycol monoethyl ether, poloxamer, glycofurol, glycerol, and mixtures thereof.

The pharmaceutical composition for topical administration may include one or more further excipients selected from the group consisting of thickening agents, gelling agents, antioxidants, fragrances, and chelating agents.

A thickening agent, viscosity-enhancing agent or gelling agent can be included to generally thicken the liquid pharmaceutical composition. While any suitable thickening agent can be included in the pharmaceutical compositions of the present invention, a preferred thickening agent, when used, includes one or more of acacia, alginic acid bentonite, carboxymethylcellulose calcium or sodium, cetostearyl alcohol, methyl cellulose, ethylcellulose, glycerin, gelatin, guar gum, hydroxyethylcellulose, hydroxypropylcellulose, hydroxypropyl-methylcellulose, locust bean gum, maltodextrin, pectin, polyacrylic acid and its derivatives (carbomers), polyvinyl alcohol, polyvinylpyrrolidone, povidone, propylene carbonate, propylene glycol alginate, sodium alginate, sodium starch glycolate, starch, highly dispersed silicium dioxide, tragacanth, tragant and xanthan gum, and any combination thereof. More preferred thickening agents are carbomer, cellulose derivatives such as sodium carboxymethyl cellulose and methyl cellulose, galactomannans such as guar gum and locust bean gum, sodium alginate, and any combination thereof. Such a thickening agent, if present, will typically form about 0.1 wt.-% to 20 wt.-%, preferably about 0.3 wt.-% to about 15 wt.-%, and more preferably about 0.5 wt.-% to 4 wt.-%, of the total weight of the pharmaceutical composition.

The pharmaceutical composition for topical administration may further contain a fragrance. Suitable fragrances include lavender oil, rose oil, lemon oil and almond oil.

In another preferred embodiment, the pharmaceutical composition according to the invention is administered intradermally, transdermally, transcutaneously or subcutaneously. In another preferred embodiment, the pharmaceutical composition according to the invention is administered intragingivally or subgingivally. In another preferred embodiment, the pharmaceutical composition according to the invention is administered intranervously. In another preferred embodiment, the pharmaceutical composition according to the invention is administered intradurally (spinally), intrathecally, or epidurally.

Preferred pharmaceutical compositions according to the invention that are preferably used for these routes of administration are described here below:

In a particularly preferred embodiment, the pharmaceutical composition according to the invention is an aqueous pharmaceutical composition, preferably an aqueous liquid, which is preferably useful for injection.

Preferably, the aqueous pharmaceutical composition comprises Tapentadol or a physiologically acceptable salt thereof; wherein
the concentration of Tapentadol is at least 1.00 mg/mL, based on the weight of Tapentadol free base and based on the total volume of the composition; and/or, preferably and
the composition comprises a buffer system; and/or, preferably and
the pH value of the composition is within the range of from 2.0 to 12, or 2.0 to 10, or 2.0 to 9.0, or 2.0 to 8.0, or 2.0 to 7.5, or 2.0 to 7.0, or 3.0 to 12, or 3.0 to 10, or 3.0 to 9.0, or 3.0 to 8.0, or 3.0 to 7.5, or 3.0 to 7.0.

Preferably, the aqueous pharmaceutical composition comprises Tapentadol or a physiologically acceptable salt thereof; wherein
the concentration of Tapentadol is at least 1.00 mg/mL, based on the weight of Tapentadol free base and based on the total volume of the composition; and/or, preferably and
the composition comprises a buffer system; and/or, preferably and
the pH value of the composition is within the range of from 2 to 12.

Preferably, the aqueous pharmaceutical composition comprises Tapentadol or a physiologically acceptable salt thereof; wherein
the concentration of Tapentadol is greater than 8.00 mg/mL, preferably at least 8.10 mg/mL, more preferably at least 9.00 mg/mL, still more preferably at least 10 mg/mL, based on the weight of Tapentadol free base and based on the total volume of the composition; and/or, preferably and
the composition comprises a buffer system; and/or, preferably and
the pH value of the composition is within the range of from greater than 3.0 to less than 6.7, preferably within the range of from 3.5 to 6.5, more preferably within the range of from 4.0 to 6.0, and most preferably within the range of from 4.5 to 6.0, or of from 4.5 to 5.5.

Preferably, the pharmaceutical composition according to the invention has undergone autoclaving, preferably at least for at least 20 minutes at least at 2 bar and at least at 121° C., and the pH value before autoclaving as well as the pH value after autoclaving preferably is independently within the range of from greater than 3.0 to less than 6.7, preferably within the range of from 4.5 to 6.0, or of from 4.5 to 5.5.

The osmolarity of the pharmaceutical composition depends on the content of its constituents and is preferably adjusted during the manufacture of the composition by the addition of an appropriate amount of an isotonizing agent, preferably sodium chloride. Other isotonizing agents such as mannitol or sorbitol can also be added alternatively or additionally. Ionic isotonizing agents are preferred.

Thus, preferably the pharmaceutical composition according to the invention comprises an isotonizing agent, more preferably sodium chloride.

Preferably, the content of the sodium chloride is not more than 1.0 wt.-%, more preferably not more than 0.8 wt.-%, still more preferably not more than 0.6 wt.-%, yet more preferably not more than 0.4 wt.-%, most preferably not more than 0.2 wt.-%, and in particular not more than 0.1 wt.-%, based on the total weight of the composition.

In preferred embodiments, the content of the sodium chloride is within the range of from 0.848±0.800 wt.-%, or 0.848±0.700 wt.-%, or 0.848±0.600 wt.-%, or 0.848±0.500 wt.-%, or 0.848±0.400 wt.-%, or 0.848±0.300 wt.-%, or 0.848±0.200 wt.-%, or 0.848±0.100 wt.-%, based on the total weight of the composition.

Preferably, the pharmaceutical composition according to the invention does not contain any preservative. For the purpose of the specification, a “preservative” preferably refers to any substance that is usually added to pharmaceutical compositions in order to preserve them against microbial degradation or microbial growth. In this regard, microbial growth typically plays an essential role, i.e. the preservative serves the main purpose of avoiding microbial contamination. As a side aspect, it may also be desirable to avoid any effect of the microbes on the active ingredients and excipients, respectively, i.e. to avoid microbial degradation. However, the pharmaceutical composition according to the invention may contain a citrate buffer system and under these circumstances, citric acid and its salts are to be considered as a buffer system and not as a preservative, though it is known that citric acid and its salt may also have a certain degree of preserving capacity.

Representative examples of preservatives include benzalkonium chloride, benzethonium chloride, benzoic acid, sodium benzoate, benzyl alcohol, bronopol, cetrimide, cetylpyridinium chloride, chlorhexidine, chlorbutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, sodium propionate, thimerosal, methyl paraben, ethyl paraben, propyl paraben, butyl paraben, isobutyl paraben, benzyl paraben, sorbic acid, and potassium sorbate.

Preferably, the pharmaceutical composition according to the invention does not contain any chelating agents such as EDTA or its sodium or calcium salts. However, the pharmaceutical composition according to the invention may contain a citrate buffer system and under these circumstances, citric acid and its salts are to be considered as a buffer system and not as a chelating agent, though it is known that citric acid and its salt also have a certain degree of chelating capacity.

Preferably, the pharmaceutical composition according to the invention does not contain any antioxidants. Examples of antioxidants that are preferably not contained in the pharmaceutical composition according to the invention include but are not limited to propyl, octyl and dodecylesters of gallic acid; butylated hydroxyanisole (BHA), butylated hydroxytoluene (BHT); ascorbic acid, sodium ascorbate; monothioglycerol; potassium or sodium metabisulfite; propionic acid; propyl gallate; sodium bisulfite, sodium sulfite; and the tocopherols or vitamin E.

Preferably, the pharmaceutical composition according to the invention contains neither any preservative nor any antioxidant.

Particularly preferred embodiments A¹ to A¹⁰ of the pharmaceutical composition according to the invention are summarized in the table here below:

| | A¹ | A² | A³ | A⁴ | A⁵ |
|---|---|---|---|---|---|
| Tapentadol HCl [mg/mL] | 23.3 ± 15 | 23.3 ± 10 | 23.3 ± 15 | 23.3 ± 10 | 23.3 ± 15 |
| buffer system [mmol/mL] | 21.4 ± 20 | 21.4 ± 10 | 13.6 ± 12 | 13.6 ± 6.0 | 6.8 ± 6.0 |
| sodium chloride [wt.-%] | 0.12 ± 0.10 | 0.12 ± 0.05 | 0.20 ± 0.18 | 0.20 ± 0.09 | 0.27 ± 0.25 |
| pH value | 5.0 ± 1.0 | 5.0 ± 0.5 | 5.0 ± 1.0 | 5.0 ± 0.5 | 5.0 ± 1.0 |
| | A⁶ | A⁷ | A⁸ | A⁹ | A¹⁰ |
| Tapentadol HCl [mg/mL] | 23.3 ± 10 | 23.3 ± 15 | 23.3 ± 10 | 23.3 ± 15 | 23.3 ± 10 |
| buffer system [mmol/mL] | 6.8 ± 3.0 | 3.4 ± 3.0 | 3.4 ± 1.5 | 1.7 ± 1.5 | 1.7 ± 0.8 |
| sodium chloride [wt.-%] | 0.27 ± 0.13 | 0.30 ± 0.28 | 0.30 ± 0.14 | 0.32 ± 0.30 | 0.32 ± 0.15 |
| pH value | 5.0 ± 0.5 | 5.0 ± 1.0 | 5.0 ± 0.5 | 5.0 ± 1.0 | 5.0 ± 0.5 |

Particularly preferred embodiments B¹ to B²⁵ of the pharmaceutical composition according to the invention are summarized in the table here below:

| | B¹ | B² | B³ | B⁴ | B⁵ |
|---|---|---|---|---|---|
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 6.3 ± 6.0 | 6.3 ± 5.0 | 6.3 ± 4.0 | 6.3 ± 3.0 | 6.3 ± 2.0 |
| pH value | 5.25 ± 0.75 | 5.25 ± 0.75 | 5.25 ± 0.75 | 5.25 ± 0.75 | 5.25 ± 0.75 |
| | B⁶ | B⁷ | B⁸ | B⁹ | B¹⁰ |
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 4.0 ± 3.5 | 4.0 ± 3.0 | 4.0 ± 2.5 | 4.0 ± 2.0 | 4.0 ± 1.5 |
| pH value | 5.25 ± 0.75 | 5.25 ± 0.75 | 5.25 ± 0.75 | 5.25 ± 0.75 | 5.25 ± 0.75 |
| | B¹¹ | B¹² | B¹³ | B¹⁴ | B¹⁵ |
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 2.0 ± 1.8 | 2.0 ± 1.6 | 2.0 ± 1.4 | 2.0 ± 1.2 | 2.0 ± 1.0 |
| pH value | 5.25 ± 0.75 | 5.25 ± 0.75 | 5.25 ± 0.75 | 5.25 ± 0.75 | 5.25 ± 0.75 |
| | B¹⁶ | B¹⁷ | B¹⁸ | B¹⁹ | B²⁰ |
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 1.0 ± 0.9 | 1.0 ± 0.8 | 1.0 ± 0.7 | 1.0 ± 0.6 | 1.0 ± 0.5 |
| pH value | 5.25 ± 0.75 | 5.25 ± 0.75 | 5.25 ± 0.75 | 5.25 ± 0.75 | 5.25 ± 0.75 |
| | B²¹ | B²² | B²³ | B²⁴ | B²⁵ |
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 0.50 ± 0.45 | 0.50 ± 0.40 | 0.50 ± 0.35 | 0.50 ± 0.30 | 0.50 ± 0.25 |
| pH value | 5.25 ± 0.75 | 5.25 ± 0.75 | 5.25 ± 0.75 | 5.25 ± 0.75 | 5.25 ± 0.75 |

Particularly preferred embodiments C¹ to C²⁵ of the pharmaceutical composition according to the invention are summarized in the table here below:

| | C¹ | C² | C³ | C⁴ | C⁵ |
|---|---|---|---|---|---|
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 6.3 ± 6.0 | 6.3 ± 5.0 | 6.3 ± 4.0 | 6.3 ± 3.0 | 6.3 ± 2.0 |
| sodium chloride [mg/mL] | 1.2 ± 1.0 | 1.2 ± 0.9 | 1.2 ± 0.8 | 1.2 ± 0.7 | 1.2 ± 0.6 |
| pH value | 5.25 ± 0.75 | 5.25 ± 0.75 | 5.25 ± 0.75 | 5.25 ± 0.75 | 5.25 ± 0.75 |
| | C⁶ | C⁷ | C⁸ | C⁹ | C¹⁰ |
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 4.0 ± 3.5 | 4.0 ± 3.0 | 4.0 ± 2.5 | 4.0 ± 2.0 | 4.0 ± 1.5 |
| sodium chloride [mg/mL] | 2.0 ± 1.8 | 2.0 ± 1.6 | 2.0 ± 1.4 | 2.0 ± 1.2 | 2.0 ± 1.0 |
| pH value | 5.25 ± 0.75 | 5.25 ± 0.75 | 5.25 ± 0.75 | 5.25 ± 0.75 | 5.25 ± 0.75 |
| | C¹¹ | C¹² | C¹³ | C¹⁴ | C¹⁵ |
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 2.0 ± 1.8 | 2.0 ± 1.6 | 2.0 ± 1.4 | 2.0 ± 1.2 | 2.0 ± 1.0 |
| sodium chloride [mg/mL] | 2.7 ± 2.4 | 2.7 ± 2.1 | 2.7 ± 1.8 | 2.7 ± 1.5 | 2.7 ± 1.2 |
| pH value | 5.25 ± 0.75 | 5.25 ± 0.75 | 5.25 ± 0.75 | 5.25 ± 0.75 | 5.25 ± 0.75 |

| | C¹⁶ | C¹⁷ | C¹⁸ | C¹⁹ | C²⁰ |
|---|---|---|---|---|---|
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 1.0 ± 0.9 | 1.0 ± 0.8 | 1.0 ± 0.7 | 1.0 ± 0.6 | 1.0 ± 0.5 |
| sodium chloride [mg/mL] | 3.0 ± 2.7 | 3.0 ± 2.4 | 3.0 ± 2.1 | 3.0 ± 1.8 | 3.0 ± 1.5 |
| pH value | 5.25 ± 0.75 | 5.25 ± 0.75 | 5.25 ± 0.75 | 5.25 ± 0.75 | 5.25 ± 0.75 |
| | C²¹ | C²² | C²³ | C²⁴ | C²⁵ |
| Tapentadol HCl [mg/mL] | 23.3 ± 15.0 | 23.3 ± 12.5 | 23.3 ± 10.0 | 23.3 ± 7.5 | 23.3 ± 5.0 |
| sodium citrate dihydrate [mg/mL] | 0.50 ± 0.45 | 0.50 ± 0.40 | 0.50 ± 0.35 | 0.50 ± 0.30 | 0.50 ± 0.25 |
| sodium chloride [mg/mL] | 3.2 ± 2.9 | 3.2 ± 2.6 | 3.2 ± 2.3 | 3.2 ± 2.0 | 3.2 ± 1.7 |
| pH value | 5.25 ± 0.75 | 5.25 ± 0.75 | 5.25 ± 0.75 | 5.25 ± 0.75 | 5.25 ± 0.75 |

Preferably, the pharmaceutical composition according to the invention has a titration acidity of not more than 1.8 mmol/L, more preferably not more than 1.7 mmol/L, still more preferably not more than 1.6 mmol/L, yet more preferably not more than 1.5 mmol/L, and most preferably not more than 1.4 mmol/L.

Preferably, the pharmaceutical composition according to the invention has a titration acidity within the range of from 1.0 to 1.8 mmol/L, more preferably 1.4 to 1.8 mmol/L.

Preferably, titration acidity is determined at a CO₂ partial pressure of 0 mm Hg under Argon at 37° C. When titrating the pharmaceutical composition according to the invention under these conditions with 0.01 M NaOH up to an endpoint of pH 7.4.

In a preferred embodiment, particularly when the composition has a pH value within the range of 5.0±0.5, the titration acidity is preferably within the range of 1.20±0.20 mmol/L, more preferably 1.20±0.10 mmol/L.

In another preferred embodiment, particularly when the composition has a pH value within the range of 4.5±0.5, the titration acidity is preferably within the range of 1.60±0.20 mmol/L, more preferably 1.60±0.10 mmol/L.

In order to satisfy high quality requirements for injection solutions, the pharmaceutical composition has to exhibit a physiologically acceptable osmolarity and a physiologically acceptable pH.

Isotonic sodium chloride solution (saline), for instance, contains 0.9 wt.-% of sodium chloride and exhibits an osmolarity of 0.308 osmol/L, which is close to the osmolarity of blood.

Preferably, the pharmaceutical composition has an osmolarity of at least 0.20 or at least 0.22 osmol/L, more preferably of at least 0.23 osmol/L, still more preferably of at least 0.24 osmol/L, yet more preferably of at least 0.25 osmol/L, most preferably of at least 0.26 osmol/L, and in particular of at least 0.27 osmol/L.

Preferably, the pharmaceutical composition has an osmolarity of not more than 0.36 osmol/L, more preferably of not more than 0.34 osmol/L, still more preferably of not more than 0.32 osmol/L, yet more preferably of not more than 0.31 osmol/L, most preferably of not more than 0.30 osmol/L and in particular of not more than 0.29 osmol/L.

In preferred embodiments, the pharmaceutical composition has an osmolarity of 0.28±0.08 osmol/L, more preferably of 0.28±0.06 osmol/L, still more preferably of 0.28±0.04 osmol/L, yet more preferably of 0.28±0.03 osmol/L, most preferably of 0.28±0.02 osmol/L, and in particular of 0.28±0.01 osmol/L.

Another aspect of the invention relates to a container comprising the pharmaceutical composition according to the invention, wherein the container is preferably a closed and airtight container. All preferred embodiments that have been defined above in connection with the pharmaceutical composition according to the invention analogously also apply to the container according to the invention.

The container according to the invention comprises a distinct volume of the pharmaceutical composition according to the invention that is adapted for administration to the patient in order to achieve local anesthesia. As the aqueous pharmaceutical composition according to the invention is typically liquid, it is preferably provided in a container. Prior to administration, the pharmaceutical composition according to the invention is then removed, completely (single dosage) or partially (multiple dosage) from the container.

Preferably, the container is a glass ampoule.

The container according to the invention may comprise a single dose of Tapentadol or may be multiple dosed. For the purpose of the specification “multiple dosed” preferably means that the container encompasses more than a single dosage unit.

In a preferred embodiment, the container contains the pharmaceutical composition according to the invention in a quantity exceeding a single administration dose (dosage unit). Under these circumstances, the container comprises multiple dosage units, i.e. is customized for more than a single administration, preferably by injection.

For example, when the container comprises a multiple dosed injection solution, its overall volume is more than the volume that is to be typically administered at once. Instead, the multiple dosed injection solution is customized for being divided into a multitude of dosage units. The individual dosage units may preferably be separated from the multiple dosage unit by means of a syringe. A typical example for a container according to the invention that comprises multiple dosage units is a preferably sterilized glass container sealed with a septum. Said glass container contains a volume of the pharmaceutical composition well exceeding the individual volume of an individual dosage unit that is intended for at once administration to the patient.

Preferably, the container contains at least 2, more preferably at least 3, even more preferably at least 5, yet more preferably at least 10, most preferably at least 12, and in particular at least 15 individual dosage units.

In another preferred embodiment, the container comprises a single dosage unit, i.e. only one individual dosage unit. Under these circumstances, according to a preferred embodiment, the container preferably comprises from 1.0 to 3.0 mL of the pharmaceutical composition. According to another preferred embodiment, the container preferably comprises from 1.0 to 500 mL, preferably 5.0 to 500 mL of the composition, e.g. 10±5 mL, or 15±10 mL, or 20±10 mL, or 25±10 mL, or 30±10 mL, or 35±10 mL, or 40±10 mL, or 45±10 mL, or 50±25 mL, or 75±25 mL, or 100±25 mL, or 150±50 mL, or 200±50 mL, or 250±50 mL, or 300±100 mL, or 400±100 mL, or 500±100 mL.

Preferably, the individual dosage units have a volume of 0.25 mL to 3.0 mL, more preferably of 0.5 mL to 2.75 mL, still more preferably of 0.75 mL to 2.5 mL, and most preferably of 1.0 mL to 2.0 mL.

In a preferred embodiment, the individual dosage units have a volume of 1.0±0.9 mL, more preferably of 1.0±0.75 mL, still more preferably 1.0±0.5 mL, yet more preferably of 1.0±0.4 mL, even more preferably of 1.0±0.2 mL, most preferably of 1.0±0.15 mL, and in particular of 1.0±0.1 mL. In another preferred embodiment, the individual dosage units have a volume of 2.0±0.9 mL, more preferably of 2.0±0.75 mL, still more preferably 2.0±0.5 mL, yet more preferably of 2.0±0.4 mL, even more preferably of 2.0±0.2 mL, most preferably of 2.0±0.15 mL, and in particular of 2.0±0.1 mL.

In still another preferred embodiment, the individual dosage units have a volume of 3.0±0.9 mL, more preferably of 3.0±0.75 mL, still more preferably 3.0±0.5 mL, yet more preferably of 3.0±0.4 mL, even more preferably of 3.0±0.2 mL, most preferably of 3.0±0.15 mL, and in particular of 3.0±0.1 mL.

According to another preferred embodiment, the individual dosage units preferably comprise from 5.0 to 500 mL of the composition, e.g. 10±5 mL, or 15±10 mL, or 20±10 mL, or 25±10 mL, or 30±10 mL, or 35±10 mL, or 40±10 mL, or 45±10 mL, or 50±25 mL, or 75±25 mL, or 100±25 mL, or 150±50 mL, or 200±50 mL, or 250±50 mL, or 300±100 mL, or 400±100 mL, or 500±100 mL.

The composition that is contained in the container may also be customized for a continual administration. Preferably, the composition that is contained in the container is adapted for a continual administration for at least 30 minutes or 45 minutes, more preferably for at least 1 h or 2 h, still more preferably for at least 3 h or 4 h, yet more preferably for at least 6 h or 8 h, most preferably for at least 10 h, and in particular for at least 12 h.

Tapentadol is administered in a locally anesthetically effective amount.

Preferably, the amount of Tapentadol that is contained in the individual dosage unit is preferably within the range of from 0.2 to 0.6 mg/kg body weight. Typically, the daily dosage of Tapentadol is within the range of from 25 mg to 600 mg, such as 25 mg, 50 mg, 75 mg, 100 mg, 150 mg, 200 mg, 250 mg, 300 mg, 400 mg, 500 mg, or 600 mg.

In a preferred embodiment, the amount of Tapentadol that is contained in the individual dosage unit is preferably within the range of from 10 mg to 250 mg, more preferably within the range of from 15 mg to 200 mg, still more preferably within the range of from 20 mg to 150 mg, yet more preferably within the range of from 30 mg to 130 mg, and most preferably within the range of from 40 mg to 115 mg, and in particular within the range of from 50 mg to 100 mg.

In another preferred embodiment, the amount of Tapentadol that is contained in the individual dosage unit is preferably within the range of from 0.1 mg to 60 mg, more preferably within the range of from 0.1 mg to 55 mg, still more preferably within the range of from 0.2 mg to 50 mg.

Preferably, the container according to the invention comprises Tapentadol in an amount within the range of from 5.0 mg to 6 g, preferably from 5.0 mg to 3 g, more preferably from 5.0 mg to 600 mg or of from 10 mg to 600 mg, based on the weight of Tapentadol free base.

In preferred embodiments, the container according to the invention comprises Tapentadol in an amount of at least 10 mg, or at least 15 mg, or at least 20 mg, or at least 25 mg, or at least 30 mg, or at least 40 mg, or at least 50 mg, or at least 75 mg, or at least 100 mg, or at least 150 mg, or at least 200 mg, or at least 250 mg, or at least 300 mg, or at least 400 mg, or at least 500 mg, or at least 600 mg, or at least 700 mg, or at least 800 mg, or at least 900 mg, or at least 1 g, or at least 1.5 g, or at least 2 g, or at least 2.5 g, or at least 3 g, or at least 3.5 g, or at least 4 g, or at least 4.5 g, or at least 5 g, or at least 5.5 g, or at least 6 g, based on the weight of Tapentadol free base.

In preferred embodiments, the container according to the invention comprises Tapentadol in an amount of at most 600 mg, or at most 550 mg, or at most 500 mg, or at most 450 mg, or at most 400 mg, or at most 350 mg, or at most 300 mg, or at most 250 mg, or at most 200 mg, or at most 175 mg, or at most 150 mg, or at most 100 mg, based on the weight of Tapentadol free base.

In preferred embodiments, the container according to the invention comprises Tapentadol in an amount of 25±15 mg, or 50±15 mg, or 75±15 mg, or 100±15 mg, or 150±15 mg, or 200±15 mg, or 250±15 mg, or based on the weight of Tapentadol free base.

The pharmaceutical composition according to the invention, particularly when it is contained in the container according to the invention, has an excellent shelf life and storage stability. Thus, preferably, the pharmaceutical composition according to the invention is preferably stable upon storage.

Preferably, the pharmaceutical composition according to the invention is stable upon storage under accelerated storage conditions at 40° C. and 75% relative humidity for at least 3 months, more preferably at least 6 months. Preferably, stability criteria are in accordance with Ph. Eur. and EMA guidelines, respectively, preferably according to the edition that is valid in February 2017.

In preferred embodiments, the pH value of the composition after storage under accelerated storage conditions at 40° C. and 75% relative humidity for at least 3 months, more preferably at least 6 months, does not relatively differ by more than ±0.4 pH units, more preferably by not more than ±0.3 pH units, more preferably by not more than ±0.2 pH units, from the initial pH value of the composition prior to storage.

Preferably, the composition is colorless before storage and after storage under accelerated storage conditions at 40° C. and 75% relative humidity for at least 3 months, more preferably at least 6 months. Preferably, the composition is colorless before storage and during/after storage, in particular during/after a storage time of more than three months, preferably of more than 6 months, more preferably of more than 12 months, most preferably of at least for twenty-four months.

In preferred embodiments, the composition has a content of decomposition products of Tapentadol after storage under accelerated storage conditions at 40° C. and 75% relative humidity for at least 3 months, more preferably at least 6 months, of not more than 1.0 wt.-%, more preferably not more than 0.9 wt.-%, still more preferably not more than 0.8 wt.-%, yet more preferably not more than 0.7 wt.-%, even more preferably not more than 0.6 wt.-%, and most preferably not more than 0.5 wt.-%, relative to the total content of Tapentadol that was originally contained in the composition prior to storage and based on the weight of Tapentadol free base. Decomposition products of Tapentadol are preferably analyzed by HPLC.

For the purpose of the specification, it may additionally be distinguished between shelf life and in-use stability. Shelf life preferably refers to the storage stability of a closed container. In-use stability preferably refers to the storage container that contains a multiple dosage unit preparation which has been utilized for the first time. Typically, the shelf life of a multiple dosage unit preparation is much longer than its in-use stability. Preferably, stability criteria are in accordance with Ph. Eur. and EMA guidelines, respectively, preferably according to the edition that is valid in February 2017.

Preferably, the pharmaceutical composition according to the invention, particularly when it is contained in the container according to the invention, exhibits a shelf life under ambient conditions of at least 6 month, more preferably at least 12 months, still more preferably at least 15 months, yet more preferably at least 18 months, most preferably at least 21 months and in particular at least 24 months.

Preferably, the pharmaceutical composition according to the invention, particularly when it is contained in the container according to the invention, is provided as a multiple dosage unit preparation that exhibits an in-use stability under ambient conditions of at least 1 week, more preferably at least 2 weeks, still more preferably at least 3 weeks, yet more preferably at least 4 weeks, most preferably at least 5 weeks and in particular at least 6 weeks.

It has been surprisingly found that the pH value can be controlled such that undesired decomposition reactions of Tapentadol can be reduced. No additional excipients are needed for stabilization.

Preferably, the pharmaceutical composition according to the invention exhibits an antimicrobial robustness that complies with the requirements of the Ph. Eur., preferably in its version for 2010. Preferably, antimicrobial robustness is achieved against S. aureus, Ps. Aeruginosa, S. spp., C. albicans, and/or A. niger, preferably satisfying the requirement of log reduction of 1, preferably 3 after 7 and no increase after 28 days. In a particularly preferred embodiment, antimicrobial robustness is achieved against bacteria satisfying the requirement of log reduction of 3 after 14 days and against molds and yeast of log reduction of 1 after 14 days.

The pharmaceutical composition according to the invention, particularly when it is contained in the container according to the invention, exhibits an excellent autoclavability, i.e. it can be subjected to autoclaving under suitable conditions for a suitable period of time without causing significant degradation of Tapentadol under the typically drastic conditions of autoclaving. Preferably, the composition is stable upon autoclaving and preferably exhibits an unaltered pH value upon autoclaving.

Preferably, the pharmaceutical composition is stable upon autoclaving for 20 minutes at 121° C. and 2 bar. Preferably, the composition is stable upon autoclaving for 20 minutes at 121° C. and 2 bar and preferably exhibits an unaltered pH value upon autoclaving under these conditions.

Preferably, stability criteria are in accordance with Ph. Eur. and EMA guidelines, respectively, preferably according to the edition that is valid in February 2017.

In preferred embodiments, the pharmaceutical composition according to the invention, particularly when it is contained in the container according to the invention, has a content of decomposition products of Tapentadol after autoclaving of not more than 0.80 wt.-%, or not more than 0.75 wt.-%, or not more than 0.70 wt.-%, or not more than 0.65 wt.-%, or not more than 0.55 wt.-%, or not more than 0.50 wt.-%, or not more than 0.45 wt.-%, or not more than 0.40 wt.-%, or not more than 0.35 wt.-%, or not more than 0.30 wt.-%, or not more than 0.25 wt.-%, or not more than 0.20 wt.-%, or not more than 0.15 wt.-%, or not more than 0.10 wt.-%, or not more than 0.75 wt.-%, or not more than 0.05 wt.-%, more preferably not more than 0.04 wt.-%, and most preferably not more than 0.03 wt.-%, relative to the total content of Tapentadol that was originally contained in the composition prior to autoclaving and based on the weight of Tapentadol free base.

In preferred embodiments, the pharmaceutical composition according to the invention, particularly when it is contained in the container according to the invention, has a content of decomposition products of Tapentadol after 10 times autoclaving, preferably in each case for 20 minutes at 121° C. and 2 bar, of not more than 4.0 wt.-%, or not more than 3.9 wt.-%, or not more than 3.8 wt.-%, or not more than 3.7 wt.-%, or not more than 3.6 wt.-%, or not more than 3.5 wt.-%, or not more than 3.2 wt.-%, or not more than 3.1 wt.-%; more preferably not more than 3.0 wt.-%, or not more than 2.9 wt.-%, or not more than 2.8 wt.-%, or not more than 2.7 wt.-%, or not more than 2.6 wt.-%, or not more than 2.5 wt.-%, or not more than 2.4 wt.-%, or not more than 2.3 wt.-%, or not more than 2.2 wt.-%, or not more than 2.15 wt.-%, or not more than 2.1 wt.-%; still more preferably not more than 2.0 wt.-%, or not more than 1.9 wt.-%, or not more than 1.8 wt.-%, or not more than 1.7 wt.-%, or not more than 1.6 wt.-%, or not more than 1.50 wt.-%, or not more than 1.4 wt.-%, or not more than 1.3 wt.-%, or not more than 1.2 wt.-%, or not more than 1.1 wt.-%; most preferably not more than 1.0 wt.-%, or not more than 0.9 wt.-%, or not more than 0.8 wt.-%, or not more than 0.75 wt.-%, or not more than 0.7 wt.-%, or not more than 0.6 wt.-%; relative to the total content of Tapentadol that was originally contained in the composition prior to first autoclaving and based on the weight of Tapentadol free base.

Preferably, the pharmaceutical composition according to the invention is for use in mammals Preferably, the mammals are humans Preferably, the humans are adults.

Preferably, the pharmaceutical composition according to the invention, particularly when it is contained in the container according to the invention, is a formulation selected from the group consisting of injection solutions, injection suspensions, and depot formulations, such as depot injection solutions, depot injection suspensions, implants and injection pumps.

Preferably, the pharmaceutical composition is an injection solution or injection suspension, which preferably is a single dosage unit form or multiple dosage unit form. Multiple dosage unit injection solutions are preferably contained in an injection vial, whereas single dosage unit forms are preferably contained in a single-use syringe.

The pharmaceutical composition according to the invention is preferably adapted for administration by injection.

Injection solutions or suspensions may be administered continuously, intermittently or patient-controlled. For the administration, injection devices such as implantable pumps, non-implantable pumps and spinal pumps may be used.

Another aspect of the invention relates to a process for the preparation of the pharmaceutical composition according to the invention or of the container according to the invention, respectively, which process comprises the step of
(a) preparing a mixture comprising Tapentadol or a physiologically acceptable salt thereof, water and a buffer system.

In a preferred embodiment, Tapentadol is employed as Tapentadol hydrochloride polymorph form A, which is preferably characterized by showing at least one or more X-ray lines (2-theta values) in a powder diffraction pattern when measured using Cu Kα radiation selected from the list comprising 15.1±0.2, 16.0±0.2, 18.9±0.2, 20.4±0.2, 22.5±0.2, 27.3±0.2, 29.3±0.2 and 30.4±0.2.

Thus, step (a) of the process according to the invention preferably comprises
the substep (a₁) of providing water for injections;
the substep (a₂) of adding buffer system, preferably sodium citrate dihydrate and dissolving;
the substep (a₃) of adding Tapentadol, preferably Tapentadol hydrochloride and dissolving;
the substep (a₄) of adding isotonizing agent, preferably sodium chloride and dissolving;
the substep (a₅) of adding acid, preferably hydrochloric acid and mixing; and
the substep (a₆) of adding further water for injections and mixing.

Substeps (a₁) to (a₆) may be performed in numerical order or in any other order.

Preferably, the process according to the invention comprises one or more additional steps selected from the group consisting of
(b) filtering the mixture through a filter, preferably of an average pore size of not more than 1.0 more preferably not more than 0.5 still more preferably not more than 0.2 μm; and/or
(c) filling the mixture into a suitable container, preferably a glass ampoule; and/or
(d) autoclaving the mixture at elevated temperature and elevated pressure, preferably at 121° C. and 2 bar for at least 20 minutes.

Preferably, steps (b), (c), and/or (d) are performed in alphabetical order.

The invention also relates to a composition or a container that is obtainable by the process according to the invention as described above.

Another aspect of the invention relates to a kit comprising the container according to the invention as described above and a packaging, wherein the container is packaged by the packaging. The container may be regarded as a primary packaging of the composition, whereas the packaging may be regarded as a secondary packaging of said primary packaging.

Thus, when the pharmaceutical composition according to the invention is contained in a container such as a glass ampoule, said container is preferably further packaged by a packaging.

Preferably, the packaging is disposable. Suitable packaging materials are known to the skilled person and include but are not limited to paper, cardboard, plastics, and metal foil. Preferably, the packaging comprises or essentially consists of cardboard.

Another aspect of the invention relates to the use of Tapentadol or a physiologically acceptable salt thereof for the preparation of a pharmaceutical composition according to the invention as described above or of a container according to the invention as described above.

Preferably, Tapentadol is employed as Tapentadol hydrochloride polymorph form A, which is preferably characterized by showing at least one or more X-ray lines (2-theta values) in a powder diffraction pattern when measured using Cu Kα radiation selected from the list comprising 15.1±0.2, 16.0±0.2, 18.9±0.2, 20.4±0.2, 22.5±0.2, 27.3±0.2, 29.3±0.2 and 30.4±0.2.

The following examples further illustrate the invention but are not to be construed as limiting its scope.

### Example 1—Tapentadol as Local Anesthetic

The local anesthetic effects of Tapentadol were studied in a guinea pig skin test. The local anesthetic effect of Tramadol was taken as a reference value.

Animals: Male Pirbright White guinea pigs from a commercial breeder (ca. 400 g body weights) were used for the study. The animals were housed under standard conditions: room temperature 22±20° C., rel. air changes per hour, air movement <0.2 m/sec, light/dark rhythm (06.00-18.00 h light, 18.00-06.00 h dark). The animals had access to water ad libitum and to an exclusive diet of “Herilan RM 204” (Eggersmann Company, Rinteln/Germany). Before the experiment they lived in groups of 5-8 animals in type IV Makrolone cages (Ebeco Company, Castrop-Rauxel/Germany). There were at least five days between delivery of the animals and the test day.

Tapentadol (test compound was) dissolved in 0.9% NaCl solution. Application route: intradermal application. Application volume: 0.1 ml per application site. Concentrations: 0.05/0.01/0.3/0.5%.

Tramadol (reference compound), was dissolved in 0.9% NaCl-solution. Application route: intradermal application. Application volume: 0.1 ml per application site. Concentrations: 0.1/0.3/1.0.

Vehicle control substance: 0.9% Na-solution was applied as a vehicle control substance by the same application route and volume as Tapentadol and Tramadol.

The experiments were performed according to a modified method originally described by Bülbring and Wajda (J Pharm Exp Ther 85: 78-84, 1945). After erasure of the back of the animals 0.1 ml of solutions of the test compounds or of vehicle (0.9% NaCl) were injected intradermally. There were 3 injection sites each on both flanks of the back with a craniocaudal distance between them of 1.5 cm. A twitch response of the skin musculature was provoked by contact of these sites with the tip of an injection cannula. The number of the stimuli needed to provoke a twitch response was registered. A maximum of 20 stimuli per site was allowed. Before the evaluation of the test solutions the sensitivity of the different skin sites was tested; in these baseline tests 1 stimulus per site was sufficient to induce the twitch response. Thereafter, solution with different concentrations of the test compounds or vehicle were injected and the skin sensitivity was evaluated at 2, 5, 10, 15 and 20 min. The different test solutions were injected on both flanks of the back into the cranial, intermediate or caudal test site in a randomized order.

Test parameter: Stimuli per twitch response.

Each concentration of the test compounds was tested at 6-7 sites. The means±s.e.m. of stimuli that were necessary to induce a twitch response were calculated for the different times after application. In addition, number of tests with a complete abolition of skin sensitivity (i.e., no twitch response after 20 stimuli) was used to calculate an EC₅₀-value (with 95% confidence limits) according to Litchfield and Wilcoxon.

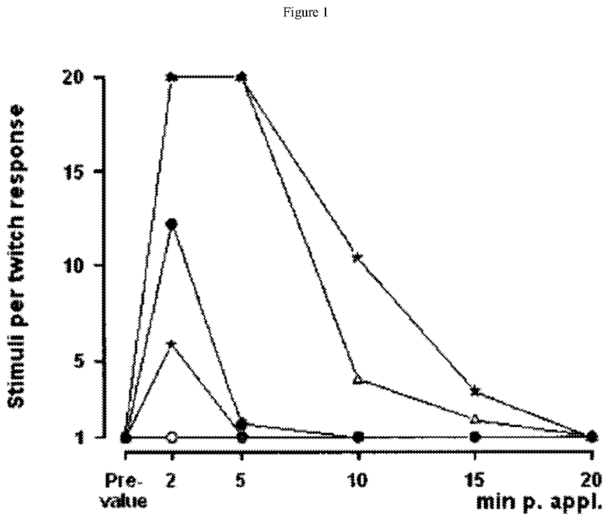

Intradermal applications of Tapentadol at concentrations from 0.05 to 0.5% exerted local anesthetic effects in guinea pigs. The number of mechanical stimuli that were necessary to evoke a skin twitch response was concentration-dependently increased by Tapentadol. At the two highest concentrations of Tapentadol (0.3 and 0.5%), a twitch response could not be elicited by 20 stimuli (the highest number of stimuli that was allowed) at 2 and 5 min after the local application of the test compound. The effect rapidly declined at 10 and 15 min after the application and completely disappeared at 20 min after the application of Tapentadol (FIG. 1, Table 1).

FIG. 1: Time- and concentration-dependent effect of Tapentadol on guinea pig skin test (local anesthetic effect). Data as mean values of n−6-7 animals per concentration level.

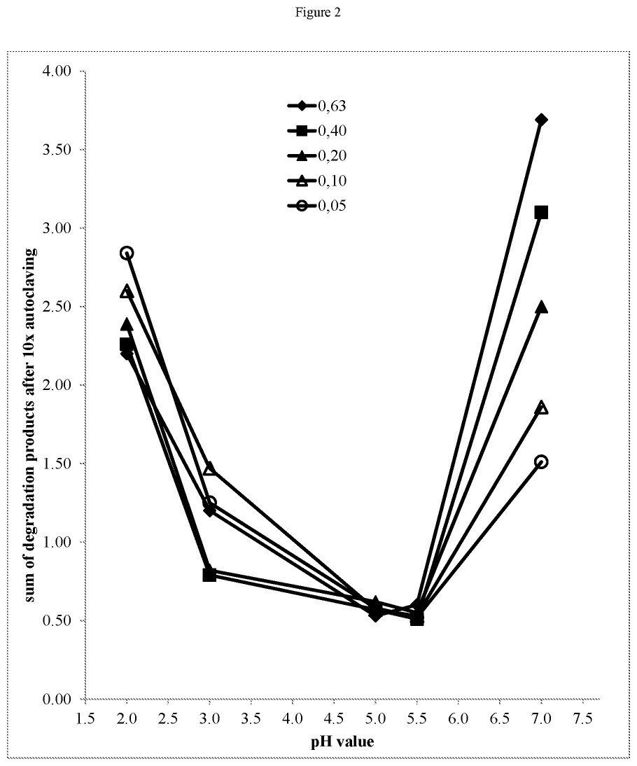

FIG. 2: pH- and wt.-% buffer-dependent effect on the sum of Tapentadol degradation products after 10 times autoclaving (♦ 0.63 wt.-% buffer, ▪ 0.40 wt.-% buffer, ▴ 0.20 wt.-% buffer, Δ 0.10 wt.-% buffer, ◯ 0.05 wt.-% buffer).

The EC₅₀-value (the concentration of test compound that abolished the skin twitch response in half of the animals) is 0.1 (0.05-0.21) % for Tapentadol. This EC₅₀-value was calculated for the maximum effect at 2 min post application:

| concentration of | Stimuli per twitch response (means ± s.e.m.) | | | | | number of animals with | EC₅₀-value |
|---|---|---|---|---|---|---|---|
| Tapentadol | Time after application [min] | | | | | abrogated twitch response | (95% CL) |
| [wt.-%] | 2 | 5 | 10 | 15 | 20 | (max. effect) | [%] |
| 0 (0.9% NaCl- | 1 | 1 | 1 | 1 | 1 | 0 | 0.1 (0.05-0.21) |
| solution) | | | | | | | |
| 0.05 | 6 ± 3 | 1 | 1 | 1 | 1 | 1 of 7 | |
| 0.1 | 12 ± 4 | 2 ± 1 | 1 | 1 | 1 | 3 of 6 | |
| 0.3 | 20 | 20 | 4 ± 3 | 2 ± 1 | 1 | 7 of 7 | |
| 0.5 | 20 | 20 | 10 ± 3 | 3 ± 2 | 1 | 7 of 7 | |

For comparison, intradermal applications of Tramadol concentration-dependently (0.1-1.0%) increased the number of mechanical stimuli that were necessary to induce a twitch response in guinea pigs. The EC₅₀-value of this local anesthetic response is 0.37 (0.29-0.48) % Tramadol:

| concentration of | Stimuli per twitch response (means ± s.e.m.) | | | | | |
|---|---|---|---|---|---|---|
| Tramadol | Time after application [min] | | | | | EC₅₀-value |
| [wt.-%] | 2 | 5 | 10 | 15 | 20 | (95% CL) [%] |
| 0.1 | 1 | 1 | 1 | 1 | 1 | 0.37 (0.29-0.48) |
| 0.3 | 8 ± 3 | 6 ± 3 | 3 ± 2 | 3 ± 2 | 3 ± 2 | |
| 1.0 | 20 | 20 | 20 | 15 ± 3 | 14 ± 3 | |

In vehicle (0.9% NaCl solution)-treated skin sites, each mechanical stimulus was able to elicit a twitch response.

These experiments therefore revealed that intradermally applied Tapentadol induces a concentration-dependent local anesthetic effect in guinea pigs. Local anesthetic effects of Tapentadol were tested in the guinea pig skin twitch response test. After intradermal applications of solutions of 0.05 to 0.5%, Tapentadol concentration-dependently increased the number of mechanical stimuli that were necessary to elicit a Skin twitch response. The maximum effect of Tapentadol was achieved at 2 min after the applications. Thereafter the effect rapidly disappeared. At 20 min p. appl., the effect had completely disappeared even after application of the highest concentration (0.5%) of Tapentadol. The EC₅₀-value (with 95% CL) for this local anesthetic effect in guinea pigs is 0.1 (0.05-0.21) % of Tapentadol. The respective value of Tramadol for this intradermal local anesthetic effect is 0.37 (0.29-0.48) %.

### Example 2—Stability of Tapentadol in Solution

A reference solution containing 15 mg/mL Tapentadol was formulated according to the following table:

| Ingredient | Content [mg/mL] |
|---|---|
| Tapentadol HCl | 17.47 |
| Sodium citrate dihydrate | 0.50 |
| Sodium chloride | 5.0 |
| Water for injections | Ad 1.003 g (1 mL) |

The pH value of this solutions was measured to be 6.7.

Different injection solutions were prepared having a concentration of Tapentadol amounting to 20 mg/mL. In each case, hydrochloric acid 1 mol q.s. was added in the amount needed in order to adjust a pH value of 2.0 (comparative), 3.0 (comparative), 5.0 (inventive), 5.5 (inventive) and 7.0 (comparative), respectively. For adjusting a pH value at buffer concentrations of 0.10 wt.-% and 0.05 wt.-%, sodium hydroxide 1 mol q.s. was added.

Batch sizes were 1000 mL, 1500 mL, 3000 mL and 50000 mL. Volumes of 2.00 mL were filled into glass ampoules.

Composition 1—buffer concentration 0.63 wt.-% (sodium citrate dihydrate, Mᵣ)—pH 2: Composition 1A, pH 3: Composition 1B, pH 5: Composition 1C, pH 5.5: Composition 1D, pH 7: Composition 1E

| per ampoule | | starting materials - | | per batch | | | | | | | | | | |
|---|---|---|---|---|---|---|---|---|---|---|---|---|---|---|
| 2 mL | | composition | wt.-% | 1000 mL | | | 3 L | | 1.5 L | | 1.5 L conc. | | 50 L | |
| 46.592 | mg | Tapentadol hydrochloride | 2.33 | 23.296 | g | 90.36 mmol | 69.888 | g | 34.944 | g | 69.888 | g | 1164.800 | g |
| 12.600 | mg | sodium citrate dihydrate. | 0.63 | 6.300 | g | 21.42 mmol | 18.900 | g | 9.450 | g | 18.900 | g | 315.000 | g |
| | | Ph. Eur. free of | | | | | | | | | | | | |
| | | endotoxins | | | | | | | | | | | | |
| 2.340 | mg | sodium chloride. Ph. Eur. | 0.12 | 1.170 | g | | 3.510 | g | 1.755 | g | 3.510 | g | 58.497 | g |
| | | free of pyrogens | | | | | | | | | | | | |
| 1938.468 | mg | water for injection * ad. | 96.92 | 969.234 | g | | 2907.702 | g | 1453.851 | g | 1407.702 | g | 48461.700 | g |
| 2000.000 | mg | | 100.00 | 1000.000 | g | | 3000.000 | g | 1500.000 | g | 1500.000 | g | 49999.997 | g |

| | | | | | |
|---|---|---|---|---|---|
| molar ratio Tapentadol:citrate = 4.22:1 | ad. | 1000.000 | g | 50000.000 | g |

Composition 2—buffer concentration 0.40 wt.-% (sodium citrate dihydrate, Mᵣ)—pH 3: pH 2: Composition 2A, Composition 2B, pH 5: Composition 2C, pH 5.5: Composition 2D, pH 7: Composition 2E

| per ampoule | | starting materials - | | per batch | | | | | | | | | | |
|---|---|---|---|---|---|---|---|---|---|---|---|---|---|---|
| 2 mL | | composition | wt.-% | 1000 mL | | | 3 L | | 1.5 L | | 1.5 L conc. | | 50 L | |
| 46.592 | mg | Tapentadol hydrochloride | 2.33 | 23.296 | g | 90.36 mmol | 69.888 | g | 34.944 | g | 69.888 | g | 1164.800 | g |
| 8.000 | mg | sodium citrate dihydrate. | 0.40 | 4.000 | g | 13.60 mmol | 12.000 | g | 6.000 | g | 12.000 | g | 200.000 | g |
| | | Ph. Eur. free of | | | | | | | | | | | | |
| | | endotoxins | | | | | | | | | | | | |
| 3.926 | mg | sodium chloride. Ph. Eur. | 0.20 | 1.963 | g | | 5.889 | g | 2.945 | g | 5.889 | g | 98.152 | g |
| | | free of pyrogens | | | | | | | | | | | | |
| 1941.482 | mg | water for injection * ad. | 97.07 | 970.741 | g | | 2912.223 | g | 1456.112 | g | 1412.223 | g | 48537.050 | g |
| 2000.000 | mg | | 100.00 | 1000.000 | g | | 3000.000 | g | 1500.000 | g | 1500.000 | g | 50000.002 | g |

| | | | | | |
|---|---|---|---|---|---|
| molar ratio Tapentadol:citrate = 6.64:1 | ad. | 1000.000 | g | 50000.000 | g |

Composition 3—buffer concentration 0.20 wt.-% (sodium citrate dihydrate, Mᵣ)—pH 2: Composition 3A, pH 3: Composition 3B, pH 5: Composition 3C, pH 5.5: Composition 3D, pH 7: Composition 3E

| per ampoule | | starting materials - | | per batch | | | | | | | | | | | |
|---|---|---|---|---|---|---|---|---|---|---|---|---|---|---|---|
| 2 mL | | composition | wt.-% | 1000 mL | | | | 3 L | | 1.5 L | | 1.5 L conc. | | 50 L | |
| 46.592 | mg | Tapentadol hydrochloride | 2.33 | 23.296 | g | 90.36 | mmol | 69.888 | g | 34.944 | g | 69.888 | g | 1164.800 | g |
| 4.000 | mg | sodium citrate dihydrate. | 0.20 | 2.000 | g | 6.80 | mmol | 6.000 | g | 3.000 | g | 6.000 | g | 100.000 | g |
| | | Ph. Eur. free of | | | | | | | | | | | | | |
| | | endotoxins | | | | | | | | | | | | | |

| | | | | | | | | | | | | | |
|---|---|---|---|---|---|---|---|---|---|---|---|---|---|
| 5.305 | mg | sodium chloride. Ph. Eur. | 0.27 | 2.653 | g | 7.958 | g | 3.979 | g | 7.958 | g | 132.635 | g |
| | | free of pyrogens | | | | | | | | | | | |
| 1944.103 | mg | water for injection * ad. | 97.21 | 972.052 | g | 2916.155 | g | 1458.077 | g | 1416.154 | g | 48602.575 | g |
| 2000.000 | mg | | 100.00 | 1000.000 | g | 3000.001 | g | 1500.000 | g | 1500.000 | g | 50000.010 | g |

| | | | | | |
|---|---|---|---|---|---|
| molar ratio Tapentadol:citrate = 13.29:1 | ad. | 1000.000 | g | 50000.000 | g |

Composition 4—buffer concentration 0.10 wt.-% (sodium citrate dihydrate, Mᵣ)—pH 2: Composition 4A, pH 3: Composition 4B, pH 5: Composition 4C, pH 5.5: Composition 4D, pH 7: Composition 4E

| per ampoule | | starting materials - | | per batch | | | | | | | | | | | |
|---|---|---|---|---|---|---|---|---|---|---|---|---|---|---|---|
| 2 mL | | composition | wt.-% | 1000 mL | | | | 3 L | | 1.5 L | | 1.5 L conc. | | 50 L | |
| 46.592 | mg | Tapentadol hydrochloride | 2.33 | 23.296 | g | 90.36 | mmol | 69.888 | g | 34.944 | g | 69.888 | g | 1164.800 | g |
| 2.000 | mg | sodium citrate dihydrate. | 0.10 | 1.000 | g | 3.40 | mmol | 3.000 | g | 1.500 | g | 3.000 | g | 50.000 | g |
| | | Ph. Eur. free of | | | | | | | | | | | | | |
| | | endotoxins | | | | | | | | | | | | | |

| | | | | | | | | | | | | | |
|---|---|---|---|---|---|---|---|---|---|---|---|---|---|
| 5.995 | mg | sodium chloride. Ph. Eur. | 0.30 | 2.998 | g | 8.993 | g | 4.496 | g | 8.993 | g | 149.876 | g |
| | | free of pyrogens | | | | | | | | | | | |
| 1945.413 | mg | water for injection * ad. | 97.27 | 972.707 | g | 2918.120 | g | 1459.060 | g | 1418.119 | g | 48635.325 | g |
| 2000.000 | mg | | 100.00 | 1000.000 | g | 3000.000 | g | 1500.000 | g | 1500.000 | g | 50000.001 | g |

| | | | | | |
|---|---|---|---|---|---|
| molar ratio Tapentadol:citrate = 26.58:1 | ad. | 1000.000 | g | 50000.000 | g |

Composition 5—buffer concentration 0.05 wt.-% (sodium citrate dihydrate, Mᵣ) -pH 2: Composition 5A, pH 3: Composition 5B, pH 5: Composition 5C, pH 5.5: Composition 5D, pH 7: Composition 5E

| per ampoule | | starting materials - | | per batch | | | | | | | | | | | |
|---|---|---|---|---|---|---|---|---|---|---|---|---|---|---|---|
| 2 mL | | composition | wt.-% | 1000 mL | | | | 3 L | | 1.5 L | | 1.5 L conc. | | 50 L | |
| 46.592 | mg | Tapentadol hydrochloride | 2.33 | 23.296 | g | 90.36 | mmol | 69.888 | g | 34.944 | g | 69.888 | g | 1164.800 | g |
| 1.000 | mg | sodium citrate dihydrate. | 0.05 | 0.500 | g | 1.70 | mmol | 1.500 | g | 0.750 | g | 1.500 | g | 25.000 | g |
| | | Ph. Eur. free of | | | | | | | | | | | | | |
| | | endotoxins | | | | | | | | | | | | | |

| | | | | | | | | | | | | | |
|---|---|---|---|---|---|---|---|---|---|---|---|---|---|
| 6.340 | mg | sodium chloride. Ph. Eur. | 0.32 | 3.170 | g | 9.510 | g | 4.755 | g | 9.510 | g | 158.497 | g |
| | | free of pyrogens | | | | | | | | | | | |
| 1946.068 | mg | water for injection * ad. | 97.30 | 973.034 | g | 2919.102 | g | 1459.551 | g | 1419.102 | g | 48651.700 | g |
| 2000.000 | mg | | 100.00 | 1000.000 | g | 3000.000 | g | 1500.000 | g | 1500.000 | g | 49999.997 | g |

| | | | | | |
|---|---|---|---|---|---|
| molar ratio Tapentadol:citrate = 53.15:1 | ad. | 1000.000 | g | 50000.000 | g |

The comparative compositions at pH 2 (i.e. compositions 1A, 2A, 3A, 4A and 5A), the comparative compositions at pH 3 (i.e. compositions 1B, 2B, 3B, 4B and 5B), the inventive compositions at pH 5 (i.e. compositions 1C, 2C, 3C, 4C and 5C), the inventive compositions at pH 5.5 (i.e. compositions 1D, 2D, 3D, 4D and 5D), and the comparative compositions at pH 7 (i.e. compositions 1E, 2E, 3E, 4E and 5E) were each autoclaved 1 time for 20 min at 2 bar and 121° C. (“1× auto”) and 10 times for 20 min at 2 bar and 121° C. (“10× auto”).

### a) Change of pH Value Upon Autoclaving

The pH values of the compositions at pH 3, 5 and 7, in each case before autoclaving (“IPC pH”), after 1 time autoclaving (“1× auto”) and after 10 times autoclaving (“10× auto”) were measured. The experimental results are compiled in the tables here below. “Δ pH 1×-10×” indicates the relative change of the pH value after 10 times autoclaving compared to 1 time autoclaving. “Δ pH IPC-10×” indicates the relative change of the pH value after 10 times autoclaving compared to the initial pH value before (any) autoclaving:

Change of pH value at pH 3:

| | sodium citrate wt.-% | IPC pH | 1x auto | 10x auto | Δ pH 1x − 10x | Δ pH IPC − 10x |
|---|---|---|---|---|---|---|
| Composition 1B | 0.63 | 3.01 | 3.07 | 3.05 | −0.02 | 0.04 |
| Composition 2B | 0.40 | 3.01 | 3.05 | 3.04 | −0.01 | 0.03 |
| Composition 3B | 0.20 | 3.00 | 3.04 | 3.06 | 0.02 | 0.06 |
| Composition 4B | 0.10 | 3.01 | 3.08 | 3.07 | −0.01 | 0.06 |
| Composition 5B | 0.05 | 3.00 | 3.08 | 3.05 | −0.03 | 0.05 |
| | mean | | | | −0.01 | 0.05 |

Change of pH value at pH 5:

| | sodium citrate wt.-% | IPC pH | 1x auto | 10x auto | Δ pH 1x − 10x | Δ pH IPC − 10x |
|---|---|---|---|---|---|---|
| Composition 1C | 0.63 | 5.01 | 4.99 | 5.01 | 0.02 | 0.00 |
| Composition 2C | 0.40 | 5.01 | 4.98 | 5.00 | 0.02 | −0.01 |
| Composition 3C | 0.20 | 5.01 | 5.00 | 5.00 | 0.00 | −0.01 |
| Composition 4C | 0.10 | 5.01 | 5.01 | 5.01 | 0.00 | 0.00 |
| Composition 5C | 0.05 | 5.00 | 4.97 | 4.98 | 0.01 | −0.02 |
| | mean | | | | 0.01 | −0.01 |

Change of pH value at pH 7:

| | sodium citrate wt.-% | IPC pH | 1x auto | 10x auto | Δ pH 1x − 10x | Δ pH IPC − 10x |
|---|---|---|---|---|---|---|
| Composition 1E | 0.63 | 7.01 | 6.95 | 6.61 | −0.34 | −0.40 |
| Composition 2E | 0.40 | 7.01 | 6.92 | 6.54 | −0.38 | −0.47 |
| Composition 3E | 0.20 | 6.99 | 6.89 | 6.39 | −0.50 | −0.60 |
| Composition 4E | 0.10 | 6.99 | 6.87 | 6.31 | −0.56 | −0.68 |
| Composition 5E | 0.05 | 7.01 | 6.73 | 6.21 | −0.52 | −0.80 |
| | mean | | | | −0.46 | −0.59 |

It can be concluded from the above experimental data that at a pH value of 7.0, at all buffer concentrations ranging from 0.05 to 0.63 wt.-%, the pH values of all compositions decreased upon autoclaving. Furthermore, it appears that at a pH value of 3.0, minor increases of the pH values were observed, whereas at a pH value of 5.0, the pH value of all compositions was robust against autoclaving.

Summing up, at all buffer concentrations, the compositions having a pH value of 5.0 showed the best stability of pH under harsh storage conditions.

### b) Change of Appearance Upon Autoclaving

The appearance of the compositions at pH 3, 5 and 7, in each case after 1 time autoclaving (“1× auto”) and after 10 times autoclaving (“10× auto”) was visually assessed. The experimental results are compiled in the tables here below:

Change of appearance at pH 3:

| | sodium citrate | | |
|---|---|---|---|
| | wt.-% | 1x auto | 10x auto |
| Composition 1B | 0.63 | clear. colorless | clear. colorless |
| Composition 2B | 0.40 | clear. colorless | clear. colorless |
| Composition 3B | 0.20 | clear. colorless | clear. colorless |
| Composition 4B | 0.10 | clear. colorless | clear. colorless |
| Composition 5B | 0.05 | clear. colorless | clear. colorless |

Change of appearance at pH 5:

| | sodium citrate | | |
|---|---|---|---|
| | wt.-% | 1x auto | 10x auto |
| Composition 1C | 0.63 | clear. colorless | clear. colorless |
| Composition 2C | 0.40 | clear. colorless | clear. colorless |
| Composition 3C | 0.20 | clear. colorless | clear. colorless |
| Composition 4C | 0.10 | clear. colorless | clear. colorless |
| Composition 5C | 0.05 | clear. colorless | clear. colorless |

Change of appearance at pH 7:

| | sodium citrate | | |
|---|---|---|---|
| | wt.-% | 1x auto | 10x auto |
| Composition 1E | 0.63 | clear. colorless | clear. light yellow |
| Composition 2E | 0.40 | clear. colorless | clear. light yellow |
| Composition 3E | 0.20 | clear. colorless | clear. light yellow |
| Composition 4E | 0.10 | clear. colorless | clear. light yellow |
| Composition 5E | 0.05 | clear. colorless | clear. light yellow |

It can be concluded from the above experimental data that at pH 7.0 at all buffer concentrations ranging from 0.05 to 0.63 wt.-%, a yellowish impurity was formed after 10 times autoclaving. Such formation could not be visually observed at pH 3.0 and pH 5.0.

Summing up, at all buffer concentrations, the compositions having a pH value of 3.0 and 5.0 showed a better stability under harsh storage conditions than the compositions at pH 7.0.

### c) Change of Assay (% LS) Upon Autoclaving

For the compositions at pH 3, 5, and 7, in each case the residual concentration of Tapentadol was determined by an assay. The measured residual concentration of Tapentadol after 1 time autoclaving (“1× auto”) and after 10 times autoclaving (“10× auto”) was determined relative to the initially adjusted concentration of 20 mg/mL. The experimental results are compiled in the tables here below. “A assay” indicates the relative change after 10 times autoclaving compared to 1 time autoclaving:

Change of assay at pH 3:

| | sodium citrate wt.-% | 1x auto | 10x auto | Δ assay |
|---|---|---|---|---|
| Composition 1B | 0.63 | 99.3% | 98.8% | 0.5% |
| Composition 2B | 0.40 | 99.6% | 99.1% | 0.4% |
| Composition 3B | 0.20 | 99.4% | 99.0% | 0.4% |
| Composition 4B | 0.10 | 99.9% | 99.3% | 0.6% |
| Composition 5B | 0.05 | 99.4% | 97.9% | 1.5% |

Change of assay at pH 5:

| | sodium citrate wt.-% | 1x auto | 10x auto | Δ assay |
|---|---|---|---|---|
| Composition 1C | 0.63 | 99.8% | 99.6% | 0.2% |
| Composition 2C | 0.40 | 99.9% | 99.8% | 0.1% |
| Composition 3C | 0.20 | 99.9% | 99.5% | 0.3% |
| Composition 4C | 0.10 | 99.9% | 99.4% | 0.5% |
| Composition 5C | 0.05 | 99.6% | 99.5% | 0.1% |

Change of assay at pH 7:

| | sodium citrate wt.-% | 1x auto | 10x auto | Δ assay |
|---|---|---|---|---|
| Composition 1E | 0.63 | 99.2% | 96.3% | 2.9% |
| Composition 2E | 0.40 | 99.5% | 98.0% | 1.5% |
| Composition 3E | 0.20 | 99.6% | 97.6% | 2.1% |
| Composition 4E | 0.10 | 99.8% | 98.2% | 1.5% |
| Composition 5E | 0.05 | 99.5% | 98.2% | 1.3% |

It can be concluded from the above experimental data that at pH 7.0, at all buffer concentrations ranging from 0.05 to 0.63 wt.-%, the residual concentration of Tapentadol decreased upon autoclaving. Furthermore, it appears that at pH values below 7.0 the stability of Tapentadol can be improved by increasing the buffer concentration. This is particularly pronounced at pH 3.0, where at a buffer concentration of 0.05 wt.-%, the residual concentration of Tapentadol relatively decreased by 1.5%, whereas at higher buffer concentrations of 0.20 wt.-% and above, the relative decrease was merely 0.4% and 0.5%, respectively.

Summing up, at all buffer concentrations, the compositions having a pH value of 3.0 and 5.0 showed a better stability under harsh storage conditions than the compositions at pH 7.0. Furthermore, increasing the buffer concentration has a stabilizing effect.

### d) Degradation (Area %) Upon Autoclaving

For all compositions, i.e. at pH 2, 3, 5, 5.5 and 7, in each case the degradation products of Tapentadol were analyzed by HPLC. The total amount of various known or unknown decomposition products of Tapentadol after 1 time autoclaving (“1× auto”) and after 10 times autoclaving (“10× auto”) was measured. The experimental results are compiled in the tables here below.

Degradation at pH 2 (Sum of all Impurities):

| comparative | sodium citrate wt.-% | 1x auto | 10x auto |
|---|---|---|---|
| Composition 1A | 0.63 | nd | 2.20 |
| Composition 2A | 0.40 | nd | 2.26 |
| Composition 3A | 0.20 | nd | 2.39 |
| Composition 4A | 0.10 | nd | 2.60 |
| Composition 5A | 0.05 | nd | 2.84 |

Degradation at pH 3 (sum of all impurities):

| comparative | sodium citrate wt.-% | 1x auto | 10x auto |
|---|---|---|---|
| Composition 1B | 0.63 | nd | 1.20 |
| Composition 2B | 0.40 | nd | 0.79 |
| Composition 3B | 0.20 | nd | 0.82 |
| Composition 4B | 0.10 | nd | 1.47 |
| Composition 5B | 0.05 | nd | 1.25 |

Degradation at pH 5 (sum of all impurities):

| inventive | sodium citrate wt.-% | 1x auto | 10x auto |
|---|---|---|---|
| Composition 1C | 0.63 | nd | 0.53 |
| Composition 2C | 0.40 | 0.05 | 0.57 |
| Composition 3C | 0.20 | 0.06 | 0.62 |
| Composition 4C | 0.10 | 0.05 | 0.57 |
| Composition 5C | 0.05 | 0.13 | 0.58 |

Degradation at pH 5.5 (sum of all impurities):

| inventive | sodium citrate wt.-% | 1x auto | 10x auto |
|---|---|---|---|
| Composition 1D | 0.63 | nd | 0.60 |
| Composition 2D | 0.40 | nd | 0.51 |
| Composition 3D | 0.20 | nd | 0.55 |
| Composition 4D | 0.10 | nd | 0.53 |
| Composition 5D | 0.05 | nd | 0.52 |

Degradation at pH 7 (sum of all impurities):

| comparative | sodium citrate wt.-% | 1x auto | 10x auto |
|---|---|---|---|
| Composition 1E | 0.63 | 0.15 | 3.69 |
| Composition 2E | 0.40 | 0.12 | 3.10 |
| Composition 3E | 0.20 | 0.06 | 2.50 |
| Composition 4E | 0.10 | 0.05 | 1.86 |
| Composition 5E | 0.05 | 0.12 | 1.51 |

The results after 10 times autoclaving for the sum of all degradation products at all tested pH values and all tested buffer concentrations are summarized in the table here below:

| | sodium | | | | | |
|---|---|---|---|---|---|---|
| | citrate | | | | | |
| | wt.-% | pH 2 | pH 3 | pH 5 | pH 5.5 | pH 7 |
| Compositions 1A to 1E | 0.63 | 2.20 | 1.20 | 0.53 | 0.60 | 3.69 |
| Compositions 2A to 2E | 0.40 | 2.26 | 0.79 | 0.57 | 0.51 | 3.10 |
| Compositions 3A to 3E | 0.20 | 2.39 | 0.82 | 0.62 | 0.55 | 2.50 |
| Compositions 4A to 4E | 0.10 | 2.60 | 1.47 | 0.57 | 0.53 | 1.86 |
| Compositions 5A to 5E | 0.05 | 2.84 | 1.25 | 0.58 | 0.52 | 1.51 |

The results are also visualized in FIG. 2 (♦ 0.63 wt.-% buffer, ▪ 0.40 wt.-% buffer, ▴ 0.20 wt.-% buffer, Δ0.10 wt.-% buffer, ◯ 0.05 wt.-% buffer).

It can be concluded from the above experimental data that within the pH range according to the invention, Tapentadol is unexpectedly stabilized against chemical decomposition. Furthermore, at comparative pH 2.0 as well as at comparative pH 7.0, the degree of chemical decomposition appears to also be a function of the buffer concentration. Unexpectedly, while at pH 2.0 the buffer seems to have a relative stabilizing effect (the higher the buffer concentration, the less degradation products are formed), at pH 7.0 the buffer seems to have an opposite relative destabilizing effect (the higher the buffer concentration, the more degradation products are formed). At inventive pH 5.0 and 5.5, however, the stability of Tapentadol against chemical decomposition is substantially improved and is not a function of the buffer concentration.

1. A pharmaceutical composition comprising Tapentadol or a physiologically acceptable salt thereof for use in local anesthesia, wherein Tapentadol or a physiologically acceptable salt thereof is the only pharmacologically active ingredient that is contained in the pharmaceutical composition.

2. (canceled)

3. (canceled)

4. The pharmaceutical composition according to claim 1, wherein local anesthesia involves a technique of local anesthesia selected from the group consisting of topical anesthesia, surface anesthesia, infiltration, plexus block, epidural (extradural) block, and spinal anesthesia (subarachnoid block).

5-7. (canceled)

8. The pharmaceutical composition according claim 1, wherein the composition is administered locally.

9. (canceled)

10. The pharmaceutical composition according to claim 1, wherein the composition is administered topically.

11. The pharmaceutical composition according to claim 10, wherein the composition is administered in form of a cream, gel, ointment, liquid, or spray.

12. The pharmaceutical composition according to claim 1, wherein the composition is administered:
topically to the skin surface, intradermally, transdermally, transcutaneously or subcutaneously;
intragingivally or subgingivally;
intranervously; or
intradurally (spinally), intrathecally, or epidurally.

13-18. (canceled)

19. The pharmaceutical composition according to claim 1, wherein the composition is in the form of an aqueous liquid.

20-26. (canceled)

27. The pharmaceutical composition according to claim 1, wherein the composition comprises a buffer system.

28-32. (canceled)

33. The pharmaceutical composition according to claim 1, wherein the pH value of the composition is within the range of from 4.0 to 6.0.

34. (canceled)

35. The pharmaceutical composition according to claim 1, wherein the pH value of the composition is within the range of from 4.5 to 5.5.

36. The pharmaceutical composition according to claim 1, wherein:
the concentration of Tapentadol is greater than 8.00 mg/mL based on the weight of Tapentadol free base and based on the total volume of the composition, and
(ii) the composition comprises a buffer system; and
(iii) the pH value of the composition is within the range of from greater than 3.0 to less than 6.7.

37-57. (canceled)

58. The pharmaceutical composition according claim 1, wherein the buffer system comprises at least one conjugate base and at least one conjugate acid, wherein said at least one conjugate base and said at least one conjugate acid independently of one another comprise one or more protonated or deprotonated acidic functional groups independently of one another selected from the group consisting of carboxylate, sulfate, sulfonate, phosphate, and phosphonate.

59. The pharmaceutical composition according to claim 1, wherein said at least one conjugate base and at least one conjugate acid are selected from the group consisting of citrate, hydrogencitrate, dihydrogencitrate and citric acid.

60-74. (canceled)

75. The pharmaceutical composition according to claim 1, wherein the total concentration of said at least one conjugate base and said at least one conjugate acid is at least 3.0 mmol/L, based on the total content of the at least one conjugate base and the at least one conjugate acid and based on the total volume of the composition.

76-115. (canceled)

116. The pharmaceutical composition according to claim 1, wherein the composition is administered by injection.

117-132. (canceled)
